(19) United States
(12) Patent Application Publication
(10) Pub. No.: US 2025/0073435 A1
(43) Pub. Date: Mar. 6, 2025

(54) SMARTSHUNT

(71) Applicant: MADISON SCIENTIFIC, INC., CHICAGO, IL (US)

(72) Inventors: Joshua MEDOW, Verona, WI (US); Bermans ISKANDER, Madison, WI (US); Michael NAGY, CHICAGO, IL (US)

(21) Appl. No.: 18/822,643

(22) Filed: Sep. 3, 2024

Related U.S. Application Data
(60) Provisional application No. 63/628,913, filed on Aug. 31, 2023.

(51) Int. Cl. A61M 27/00; A61M 39/26

(52) U.S. Cl. CPC A61M 27/006; A61M 39/26; A61M 2039/265; A61M 2205/18; A61M 2205/3327; A61M 2205/3331; A61M 2205/3592; A61M 2205/50; A61M 2205/8206; A61M 2205/8243; A61M 2210/0687; A61M 2230/62

(57) ABSTRACT

A cranial shunt implantable into a patient, the cranial shunt may comprise a first catheter, a second catheter and a valve assembly operatively coupled with the first and second catheters, the valve assembly comprising an inlet and a microcontroller, wherein the first catheter transfers cerebrospinal fluid (CSF) to the inlet. The cranial shunt may also comprise a pressure sensor configured to provide intracranial pressure (ICP) of the patient to the microcontroller, and a tilt sensor configured to provide an angle of orientation relative to gravity of a cranium of the patient to the microcontroller, where the valve assembly passes or blocks the CSF flow to the second catheter based on instructions from the microcontroller.

### CROSS REFERENCE TO RELATED APPLICATIONS

The application is a continuation in part of PCT Application No. PCT/US23/024582 filed on Jun. 6, 2023, entitled “SMARTSHUNT,” which claims priority to priority to and benefit of U.S. Provisional Patent Application No. 63/349,374, filed on Jun. 6, 2022, entitled “CEREBROSPINAL FLUID SHUNT ASSEMBLY, SYSTEM AND METHOD,” and U.S. Provisional Patent Application No. 63/457,164, filed on Apr. 5, 2023, entitled “SMARTSHUNT,” each of which are incorporated herein by reference in their entirety. This application also claims priority to U.S. Provisional Patent Application No. 63/628,913, filed on Aug. 31, 2023, which is incorporated herein by reference.

### TECHNICAL FIELD

The present teachings relate to a shunt, and more particularly to a smart shunt system utilized for hydrocephalus.

### BACKGROUND

Cerebrospinal Fluid (CSF), which resembles blood serum, surrounds the brain and spinal column to act as a mechanical shock absorber and to drain residual particles. Structures in the brain produce CSF, and other structures drain it into the venous system at a rate of approximately 500 mL per day in a healthy adult. In hydrocephalus patients, overproduction and/or underdrainage causes a buildup of CSF in the cranium. Hydrocephalus can significantly increase pressure inside the cranium, leading to severe headaches, skull deformation, cognitive impairment, brain damage, or death. If left untreated, 90% of pediatric patients will die before age 10, and survivors will be cognitively impaired. One person in 700 is born with hydrocephalus. Others may develop it later due to traumatic head injury, cerebral hematoma, infection, or due to aging (‘Normal Pressure Hydrocephalus’).

For the past 60 years, cranial shunts have been the standard of care for hydrocephalus. The proximal catheter is a short tube inserted through a burr hole in the skull to a drainage point in the cranium. The proximal (‘proximal’=towards the brain's center) end of the proximal catheter has holes to drain CSF that is pushed into them by the high pressure in the cranium. The distal end connects to a low profile valve, located outside the skull underneath the skin and muscle layers of the scalp.

The valve is generally a simple mechanical spring device that is closed when the pressure across it (delta pressure or ‘ΔP’) is low but allows flow in proportion to pressure as ΔP increases. The valve usually includes a separate check valve that blocks reverse flow into the brain, in cases where ΔP is negative (distal pressure higher than proximal pressure). The valve may also include a “reservoir,” a flat cylinder with a flexible diaphragm on top made of self-sealing silicone or similar material that allows puncture with a needle to draw CSF samples or inject drugs. The valve conducts the CSF downstream to the distal catheter (‘distal’=away from the brain), a long flexible tube that passes subcutaneously under the scalp, then through the thorax to a location into which the CSF will be drained and absorbed by the body. As many shunts are implanted in pediatric or neonate patients, extra catheter length is often provided during implantation to allow for future patient growth.

The most common type of hydrocephalus shunt is the Ventriculoperitoneal (VP) Shunt, in which the proximal catheter is tunneled through the brain to one of the larger brain ventricles, which are openings deep within the brain that contain CSF. The distal catheter extends all the way down to the patient's upper abdomen where it terminates in the peritoneum. For patients with peritoneal complications, drainage into the right atrium of the heart or the pulmonary pleura are sometimes indicated.

Alternative hydrocephalus treatment methods, such as lumbar shunts, structural brain surgeries, and endoscopic third ventriculoscopy (ETV), are performed for a minority of special cases where a VP shunt may be contraindicated. Another prior art device, drains from a subarachnoid space in the posterior fossa into a branch of the internal jugular vein, and is implanted using a venous catheter.

In general, proximal catheters, valves, and distal catheters come in standard sizes and different manufacturers' products can be used together. Currently marketed shunts come in different valve configurations. “Delta Pressure” (DP) type valves account for 70% of the market, have a fixed opening ΔP and operate along a single pressure/flowrate characteristic curve. A small subset of the DP valves are “Constant Flowrate” valves, which behave like DP valves at lower ΔP, but at midrange ΔP provide a constant flowrate regardless of ΔP. At high ΔP, the Constant Flowrate valve opens fully and follows a steep linear pressure-flowrate relationship. “Programmable Valves”, in contrast, can be reset non-invasively by a physician to operate along different pressure-flowrate curves. This is generally accomplished noninvasively using a permanent magnet in the implanted valve that is rotated or translated using a magnet outside the skin to change the valve's flow resistance.

Current shunts offer various features, including proximal catheters with antibiotic coatings to prevent infection, fluoroscopic markings to aid navigation and inspection, and anti-siphon devices (ASDs). ASDs are mechanical gravity-driven fluid switches positioned downstream of the valve, near the top of the distal catheter. Their purpose is to counter the “siphon effect”, in which the fluid column weight of CSF in the distal catheter suddenly lowers the pressure at the top of the distal catheter when the patient moves from a recumbent to an upright position. If the movement is sudden, the siphon effect can cause a large amount of CSF to quickly shoot through the valve, over draining the cranium and leading to acute or long-term problems. When the patient is recumbent, the ASD channels CSF through a larger, low-resistance opening to allow high flow, but when the patient is upright, the ASD's mechanical gravity switch blocks the larger opening and CSF flows through a smaller, high-resistance opening at low flowrate.

Approximately 125,000 Americans of all ages have permanent shunts implanted. Forty thousand hydrocephalus surgeries take place in the US each year, most of the shunt implantations are revisions. Ninety percent of children with shunts will survive to adulthood, and the majority of those will have normal cognitive development.

VP shunts suffer from high failure rates. Forty percent fail within the first year, and eighty percent within the first three years of implantation. Obstruction of the proximal catheter accounts for 72% of these failures. Obstruction failures put the patient at great medical risk and require revision surgery. A growing body of recent research indicates that over drainage of CSF may be a primary root cause of obstruction. Over drainage can cause the brain's ventricles to shrink in size, causing brain tissue to be drawn into the proximal catheter's drainage holes, resulting in blockage. Over drainage, in turn, may be caused by: siphoning caused by a patient's sudden posture change from recumbent to upright, high pressure transients in intra cranial pressure (ICP) due to Valsalva maneuvers such as shouting, straining, coughing, etc., inability to properly set the pressure drop needed for drainage in a conventional valve, due to system inaccuracies and incomplete knowledge of a patient's actual gauge ICP.

Therefore, a permanently implantable hydrocephalus shunt is needed that can reliably maintain normal ICP without overtraining. Additional features such as on-demand ICP readings, data logging, remote noninvasive calibration, anti-siphoning, precise pressure regulation, clog detection, and self-test, are highly desirable.

### SUMMARY

The following presents a summary of this disclosure to provide a basic understanding of some aspects. This summary is intended to neither identify key or critical elements nor define any limitations of embodiments or claims. Furthermore, this summary may provide a simplified overview of some aspects that may be described in greater detail in other portions of this disclosure. This summary is intended to include various combinations of described aspects.

A cranial shunt implantable into a patient, the cranial shunt may comprise a first catheter, a second catheter and a valve assembly operatively coupled with the first and second catheters, the valve assembly comprising an inlet and a microcontroller, wherein the first catheter transfers cerebrospinal fluid (CSF) to the inlet. The cranial shunt may also comprise a pressure sensor configured to provide intracranial pressure (ICP) of the patient to the microcontroller, and a tilt sensor configured to provide an angle of orientation relative to gravity of a cranium of the patient to the microcontroller, where the valve assembly passes or blocks the CSF flow to the second catheter based on instructions from the microcontroller.

The above cranial shunt may comprise any of the foregoing in any combination:
an external charging device and a power subsystem, wherein the power subsystem comprises a rechargeable electromechanical energy storage cell and a power management circuit for wireless charging by the external charging device.
a control subsystem and a power subsystem, wherein the microcontroller sends status data of the power subsystem to the control system.
a communication subsystem, wherein the control subsystem sends commands and data to and receives data from, the communications subsystem.
an external device, wherein the communications subsystem communicates wirelessly with the external device.
a first antenna and a second antenna.
an external charging device and a power subsystem, wherein the power subsystem comprises a rechargeable electromechanical energy storage cell.
where the first antenna resides on a printed circuit board (PCB) or within a component inside the electronics box and the second antenna is a single loop track that encircles the perimeter of the valve assembly.
where the first and second antennas operate at spaced frequencies so charging of the rechargeable electromechanical energy storage cell and communication functions of the microcontroller can take place simultaneously for uninterrupted operation during charging of the rechargeable electromechanical energy storage cell.
where a charging frequency of the rechargeable electromechanical energy storage cell and any subharmonics resulting from charging are outside a range of human hearing.
where the pressure sensor is attached to a tube, and measures intra-cranial pressure in a subarachnoid space within the cranium, or inside a brain's parenchyma.
where the pressure sensor measures ICP at a location outside a fluid channel connected to the valve assembly.
where if the valve assembly is open for a specified time and ICP fails to decrease by a preselected threshold amount, an external device alerts the patient the first catheter may be clogged.
an external device, wherein the external device provides an ambient pressure reading to the microcontroller.
where the microcontroller uses the ambient pressure reading to adjust the ICP based on the patient's environmental surroundings.
where the external device comprises a wearable device.
where the wearable device comprises a wrist worn device.

A medical device may comprise an implanted catheter, an implanted valve assembly operatively coupled with the catheter, the valve assembly comprising an inlet and a microcontroller, wherein the catheter transfers cerebrospinal fluid (CSF) to the inlet, an implanted pressure sensor configured to provide intracranial pressure (ICP) of the patient to the microcontroller, wherein the valve assembly passes or blocks the CSF based on instructions from the microcontroller and an implanted rechargeable electromechanical energy storage cell operatively coupled with the microcontroller. The medical device may further comprise an external charging device configured to charge the rechargeable electromechanical energy storage cell wirelessly through derma, an external communication device in wireless data communication with the microcontroller, wherein the external device is configured to receive and send information from and to the microcontroller, an external docking station configured to charge rechargeable batteries located within said external charging device and said external device and a clinical software application installed on a device, the clinical application in communication with the external communication device.

The above medical device may comprise any of the foregoing in any combination:
where the information received and sent by the external communication device comprises ambient pressure of an environment of the patient
where the external communication device provides alerts indicating a potentially undesirable condition for the patient, wherein such alerts are communicated to the external communication device wirelessly from the microcontroller.
where the undesirable condition comprises a possible blockage in the catheter;
where the external communication device provides a measurement of ambient pressure to the microcontroller.
where the external communication device is configured to be stored on the docking station.
where the microcontroller uses the ambient pressure reading to adjust the ICP based on the patient's environmental surroundings.
where the external device comprises a wearable device.
where the wearable device comprises a wrist worn device.

### DESCRIPTION OF THE DRAWINGS

The present teachings may be better understood by reference to the following detailed description taken in connection with the following illustrations, wherein:

FIG. 1 is a cross-sectional view of an embodiment of an implant applied in a patient;

FIG. 2 is a functional block diagram of an embodiment of a smartshunt system depicting the relationships and subsystems of the smartshunt system and components thereof;

FIG. 3 is a conceptual depiction of an embodiment of the components and subsystems of a smartshunt system, including example embodiments and uses;

FIG. 4 is a functional block diagram of an embodiment of a valve assembly of a smartshunt system depicting embodiments of components thereof;

FIG. 5A is a perspective view of an embodiment of an implant and a valve assembly;

FIG. 5B is a top view of another embodiment of an implant and a valve assembly;

FIG. 5C is a side view of the implant and valve assembly of FIG. 5B;

FIG. 5D is a perspective view of the implant and valve assembly of FIG. 5B;

FIGS. 6A-B are top and enlarged perspective views of an embodiment of a proximal catheter, also showing methods for application;

FIGS. 7A-C top and cross-sectional views of an embodiment of a proximal catheter, including single lumen and dual lumen catheter sections;

FIG. 8A is a perspective view of an embodiment of an implant and a valve assembly;

FIGS. 8B-H shows embodiments of a pressure capsule;

FIG. 9 shows embodiments of different placement locations of a pressure sensor of an implant in a smartshunt system;

FIG. 10 is a perspective view of an embodiment of an implant and a valve assembly;

FIG. 11 is a perspective view of an embodiment of an implant and a valve assembly;

FIGS. 12A-B show perspective views of an embodiment of an implant and a valve assembly in a single-box configuration;

FIG. 13 is a functional block diagram of an embodiment of a valve assembly of a smartshunt system depicting embodiments of components thereof;

FIG. 14 is a perspective view of an embodiment of an implant and a valve assembly;

FIGS. 15A-B are side and top views showing an embodiment of a charger as selectively coupled to the implant (and antenna of the implant) for charging of the system;

FIGS. 16A-B shows various embodiments of a charger;

FIGS. 17A-B show an embodiment of the influence of fluid column and brain weight on pressure accuracy; FIG. 17C shows an embodiment of a brain map for calibration;

FIGS. 18A-B are flow diagrams showing embodiments of timed reading, sleeping, and recharging states of an implant;

FIG. 19 is a flow diagram showing an embodiment of off-state modes of an implant;

FIG. 20 is a flow diagram showing an embodiment of timed-reading of an implant;

FIG. 21 is a flow diagram showing an embodiment of timed-reading of an implant;

FIG. 22 is a flow diagram showing an embodiment of low power modes of an implant;

FIG. 23 is a flow diagram showing an embodiment of sleep modes of an implant;

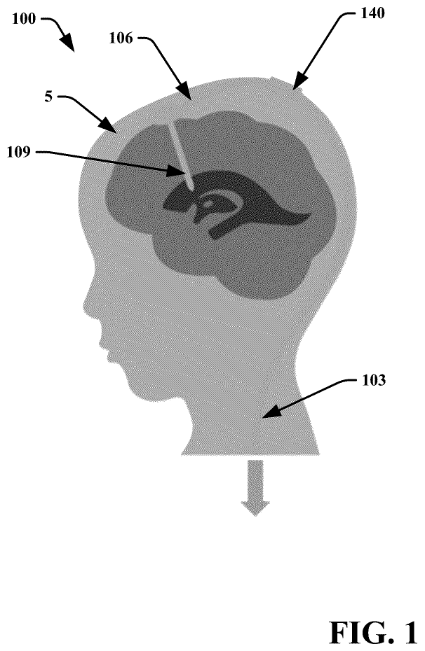

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DETAILED DESCRIPTION

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

Reference will now be made in detail to embodiments of the present teachings, examples of which are illustrated in the accompanying drawings. It is to be understood that other embodiments may be utilized, and structural and functional changes may be made without departing from the scope of the present teachings. Moreover, features of the embodiments may be combined, switched, or altered without departing from the scope of the present teachings, e.g., features of each disclosed embodiment may be combined, switched, or replaced with features of the other disclosed embodiments. In this disclosure, numerous specific details provide a thorough understanding of the subject disclosure. It should be understood that aspects of this disclosure may be practiced with other embodiments not necessarily including all aspects described herein, etc. As such, the following description is presented by way of illustration and does not limit the various alternatives and modifications that may be made to the illustrated embodiments and still be within the spirit and scope of the present teachings.

As used herein, the words “example” and “exemplary” mean an instance, or illustration. The words “example” or “exemplary” do not indicate a key or preferred aspect or embodiment. The word “or” is intended to be inclusive rather an exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggest otherwise.

Throughout this disclosure, ‘battery’ and ‘cell’ are used interchangeably, regardless of the number of electrochemical energy storage cells in the unit. ‘Proximal’ means towards the brain, ‘distal’ away from the brain.

A ventriculoperitoneal embodiment means a proximal catheter drains from the ventricle and a distal catheter empties into the abdominal peritoneum. However, other configurations, such as drainage from the subarachnoid space, or discharge into the right atrium or pleural sac, are also possible based on the present teachings. The present disclosure is not limited to the location of discharge. What is described herein is exemplary and any appropriate location of discharge within our even outside of the body may be utilized without departing from the present teachings.

In an embodiment disclosed, the smartshunt system 1000 may comprise five separate medical devices that work together to achieve the intended functions. It should be understood, however, that additional medical devices may be added without departing from the present teachings. Moreover, configurations of the medical devices may be altered or even combined without departing from the present teachings.

The present embodiment of the smartshunt may comprise a device to maintain ICP below a physician selectable threshold by conducting excess CSF from the cranium to another body location such as the abdominal peritoneum. The present embodiment of the smartshunt may comprise a device to reduce overdrainage by draining only when average ICP is within its acceptable limit, and not in response to transient pressure spikes or changes due to posture.

The present embodiment of the smartshunt may comprise a device to provide on-demand ICP readings to the patient anytime, anywhere, as well as status alerts such as low battery and possible catheter obstruction. The present embodiment of the smartshunt may comprise a device that provides physicians with convenient, noninvasive device access during office visits for longitudinal data download, settings adjustment, and device re-calibration. Further, the present embodiment of the smartshunt may comprise a device that provides physicians with longitudinal data that presents a detailed record of patient ICP with numerous timestamped readings every day, all day. As noted, these devices may comprise many different configurations to accommodate these features. Moreover, a single device may include one or more of these features.

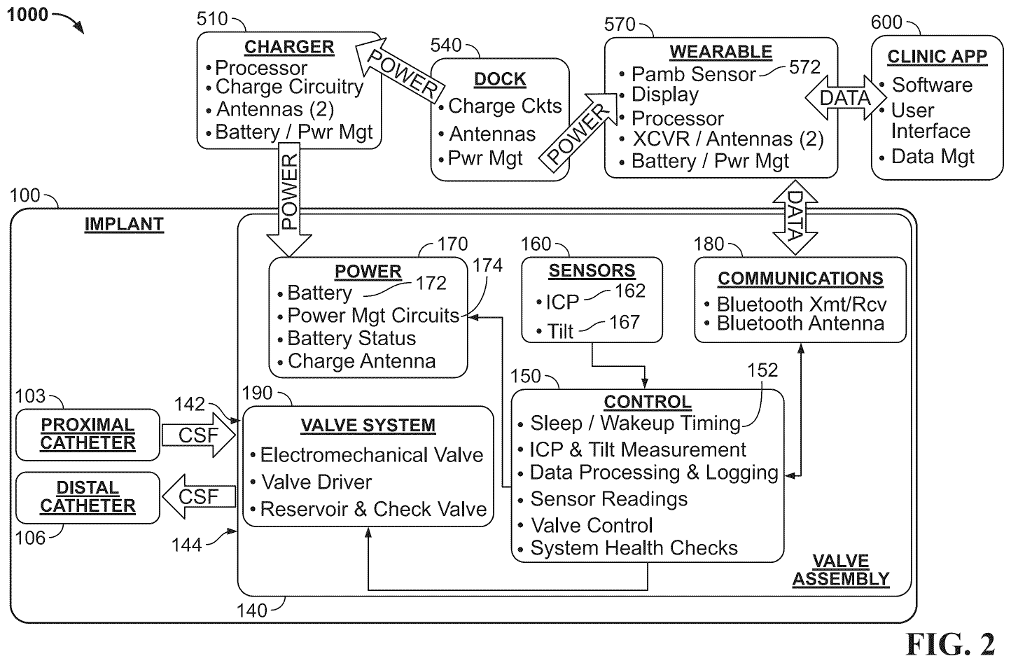

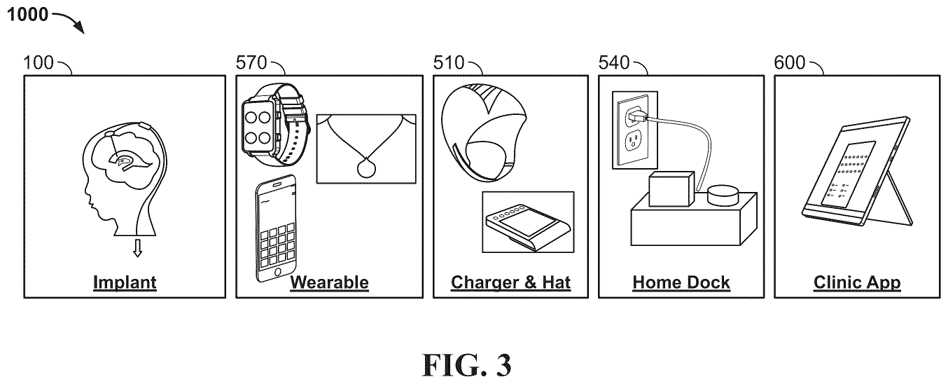

The smartshunt system 1000 may comprise the following as shown in FIG. 2. Shown in FIG. 2 is an implant 100, comprised of a proximal catheter (also referred to as a first catheter) 103, distal catheter (also referred to as a second catheter) 106, and valve assembly 140. The proximal catheter 103 transfers Cerebrospinal Fluid (CSF) to an inlet 142 of the valve assembly 140, which passes or blocks flow out to the distal catheter 106, through an outlet 144, which shunts the CSF to the abdominal peritoneum.

Inside the valve assembly 140, a control subsystem 150 including, in an example, microcontroller 152 sends control commands to operate other subsystems within the implant 100. The microcontroller 152 may also receive data from other subsystems within the implant 100, such as sensor data from an intracranial pressure (ICP) sensor 162 and a tilt sensor 167, which may comprise a sensor subsystem 160.

The implant 100 may comprise a power subsystem 170. The power subsystem 170 may comprise a rechargeable electromechanical energy storage cell or battery 172 and a power management circuit 174 or circuits for wireless recharge by an external charger device 510. The power subsystem 170 provides power to all other subsystems inside the valve assembly 140, and receives commands and sends status data to other subsystems in the implant 100 or system 1000. For example, the power subsystem 170 may send data like the current battery charge to a control subsystem 150 of the implant 100. The control subsystem 150 may send commands and data to, and receives data from, a communications subsystem 180 that communicates wirelessly with a wearable device 570, which may similarly communicate and receive/transfer data to a clinic app 600, and the like. The control subsystem 150 may send commands and data to, and receives data from, other subsystems and components of the implant 100 and system 1000, including from the valve subsystem 190, and the like. FIG. 2 shows an embodiment of potential communications and data or power transfer between subsystems and components of the implant 100 and system 1000.

The wearable device 570 may provide the implant 100 with ambient pressure data (e.g., through a pressure ambient sensor 572) and physician provided adjustable settings and may receive data uploads from the implant 100 after each implant measurement cycle, which is governed by an internal timer 152. The smartshunt system 1000 may comprise a dock device 540 configured to plug into a power source, such as a wall outlet in a patient's home. The dock device 540 may wirelessly recharge the wearable device 570 and charger device 510 batteries. The charger device 510 may then be used to charge the implant 100. The smartshunt system 1000 may further comprise a clinic application 600 that may be installed on any appropriate device, i.e., the clinical application may comprise a software application that resides on a third-party laptop, tablet, smart watch, or phone device. It may be used by physicians to wirelessly upload data from and change settings in the implant 100, via the wearable device 570 and to generally review and manage patient care, trends, and outcomes.

The proximal catheter 103 may be of any appropriate configuration. In the embodiment shown, the proximal catheter 103 may comprise an outer diameter 2.5 mm and inner diameter of 1.5 mm (within a tolerance of 0.2 mm). The proximal catheter 103 may comprise a flexible tube 113 with drain holes 119 in its proximal end 116. The proximal catheter 103 may be configured to tunnel through the brain parenchyma through a burr hole in the skull 5 of the patient. The proximal end 116 of the proximal catheter 103 may reside permanently in the ventricle of the patient, where it drains CSF. The proximal catheter 103 may comprise a tapered, open proximal tip to accommodate an optional ventriculoscope during implantation. The proximal catheter 103 may couple with or branch into a sensing lumen 101. In some embodiments, the proximal catheter 103 may be a commonly available size, allowing physicians to use many existing third party catheters, which may simplify surgical replacement of existing shunts with the smartshunt system 1000.

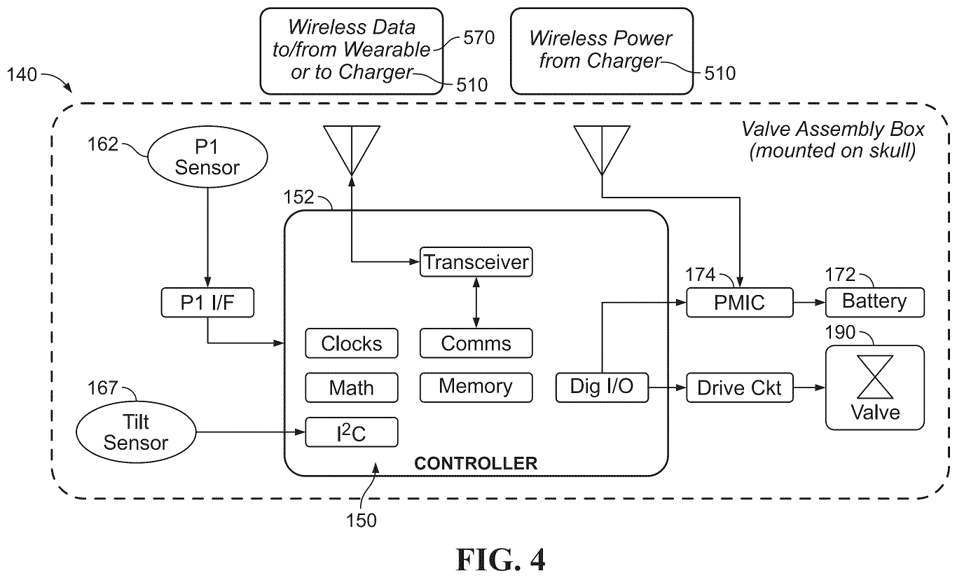

The valve assembly 140, a block diagram of which is shown in FIG. 4, is a small assembly that resides on top of the skull, under the scalp of the patient once implanted. The valve assembly 140 may comprise an electromechanical latching valve based on shape memory alloy technology to provide silent switching, small size, and low power. The valve assembly 140 may comprise a low-drift, media compatible pressure sensor 162 configured to measure absolute ICP, and a 3-axis chip accelerometer used as a tilt sensor 164 configured to measure the patient's posture relative to gravity. This measurement may help to compensate for fluid column weight in the proximal catheter 103 and in the cranium, and to detect siphoning events through the distal catheter 106. The microcontroller 152 may control the operating sequence, processes measurements, and control the valve assembly 140 state (open or closed). A wireless Bluetooth Low Energy (BLE) transceiver circuit in the valve assembly 140 may allow wireless communications with the external wearable device 570. The valve assembly 140 may be powered by a battery 172, such as a rechargeable high-density lithium coin cell. Wireless charging and power management circuitry may be included in the valve assembly 140 to ensure safe, reliable recharge from the external charger device 510.

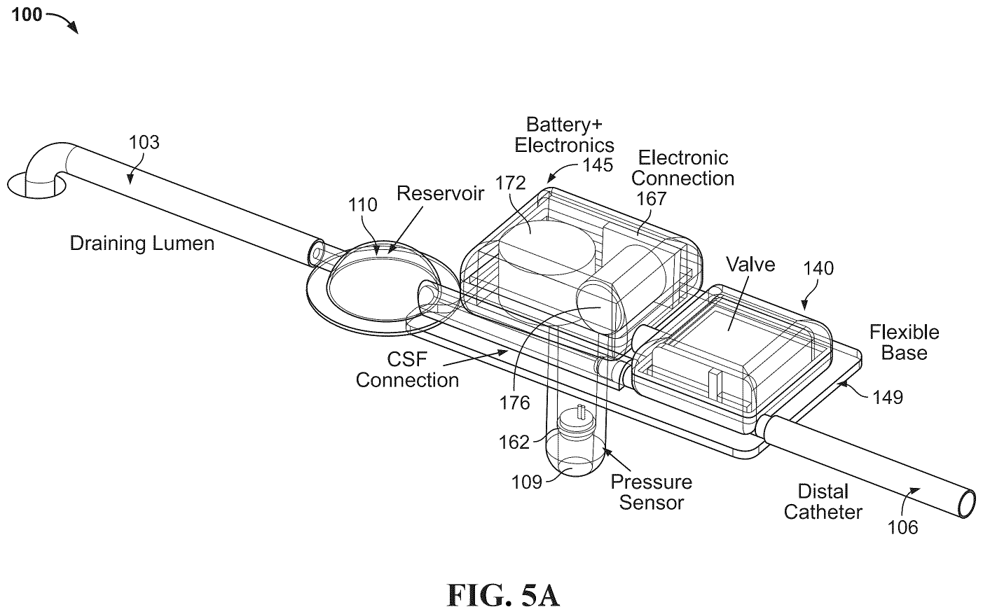

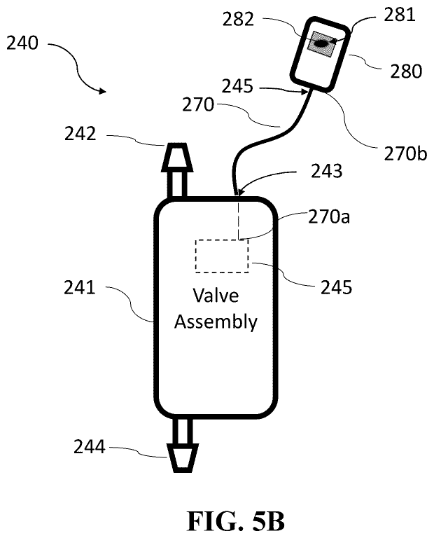

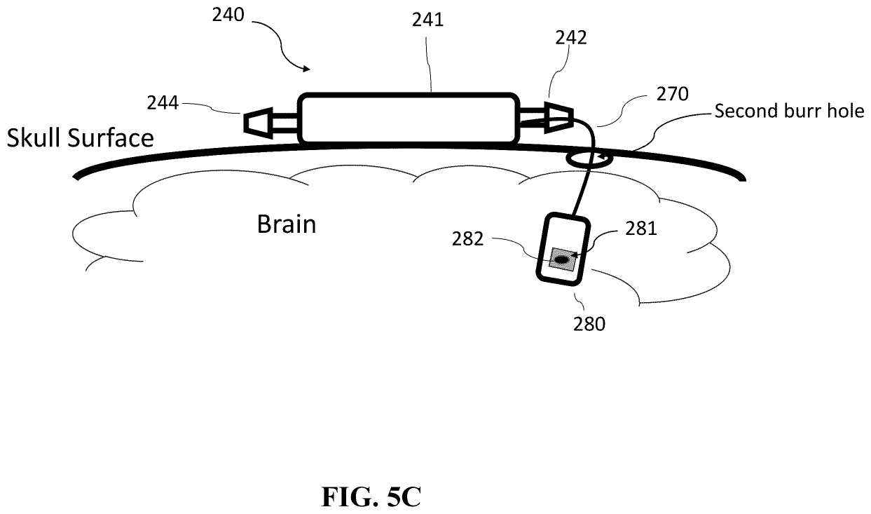

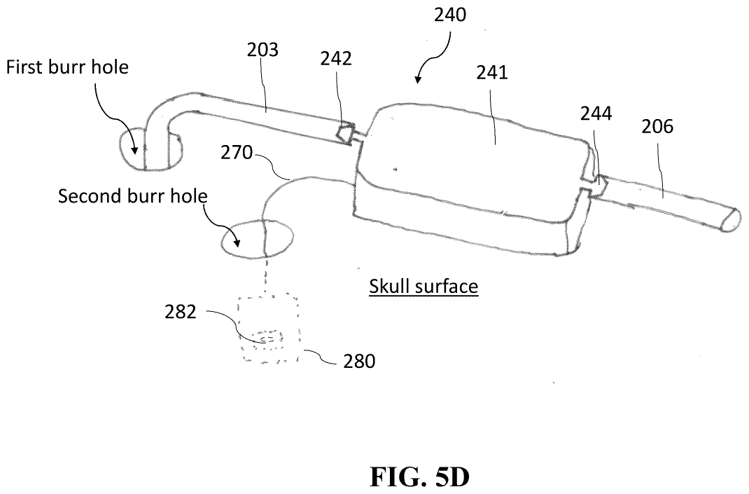

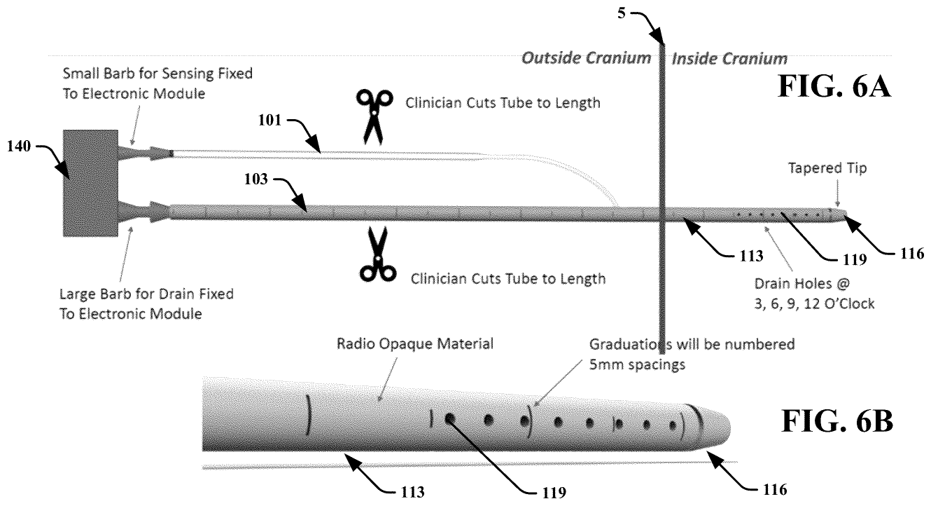

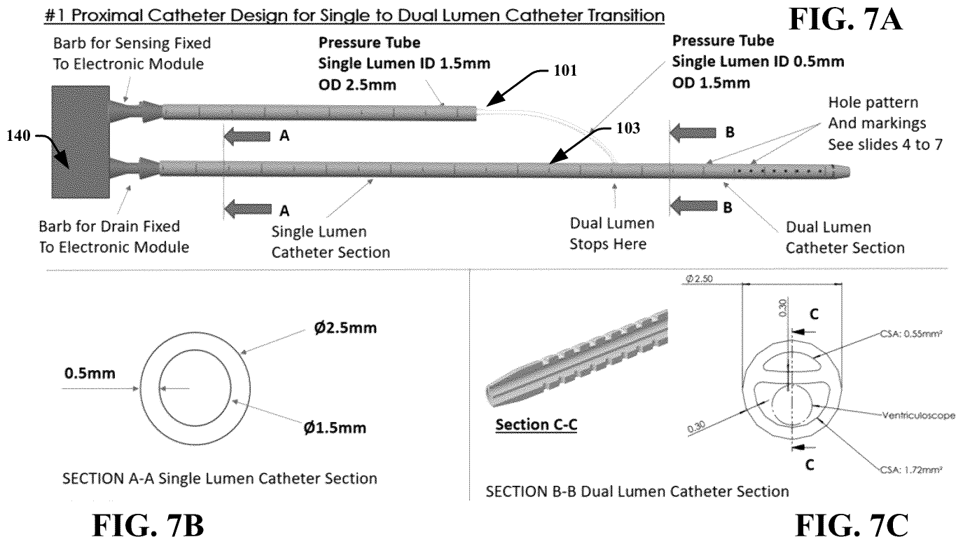

FIG. 5A illustrates an embodiment of the valve assembly's 140 physical layout. CSF enters from the left via the proximal catheter 103. A semi-permeable, self-sealing silicone reservoir 110 may be included such that it may be accessed through the scalp by a needle for CSF sampling or drug injection. The reservoir 110 is an optional and may be placed in the fluid path if desired. From the reservoir 110, the CSF enters the valve assembly 140, or more specifically as shown the rectangular box in the middle of the valve assembly 140, and exits the valve assembly 140 to the right into the distal catheter 106. To the left of the valve assembly 140 and connected by a three-wire electrical cable is an electronics box 145, which may contain the battery (e.g., power subsystem 170) and all electronic components (e.g., control subsystem 150). The electronics box 145 may include a battery or coin cell 172 and a large super-capacitor 176 used to supply peak current to the valve assembly 140 during state change. Protruding from the bottom of the electronics box is a rigid tube 109 that extends into a second burr hole (the first burr hole being for the proximal catheter 103). The pressure sensor 162 may be welded into the end of the rigid tube 109, and measures ICP in the subarachnoid space just below the dura, or somewhat deeper, inside the brain's parenchyma.

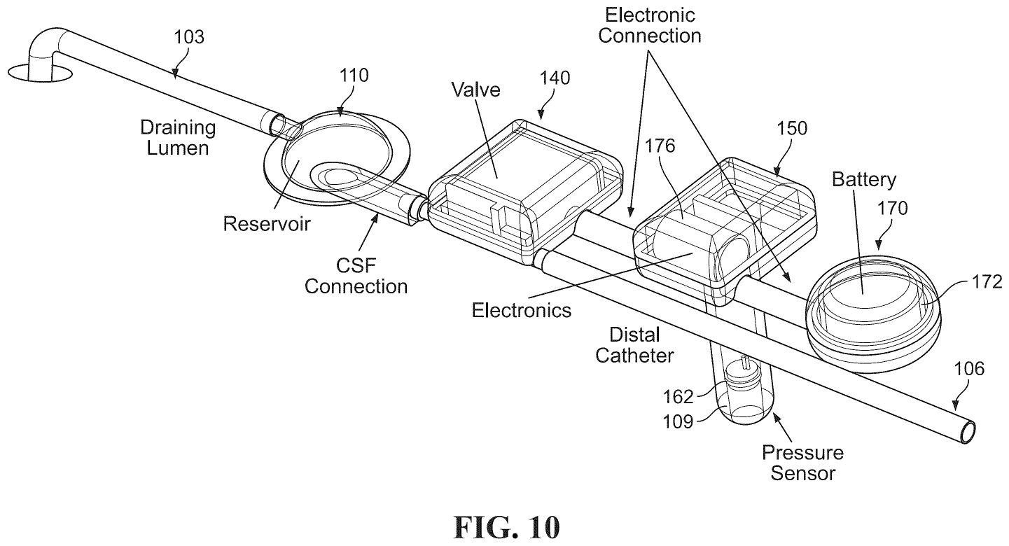

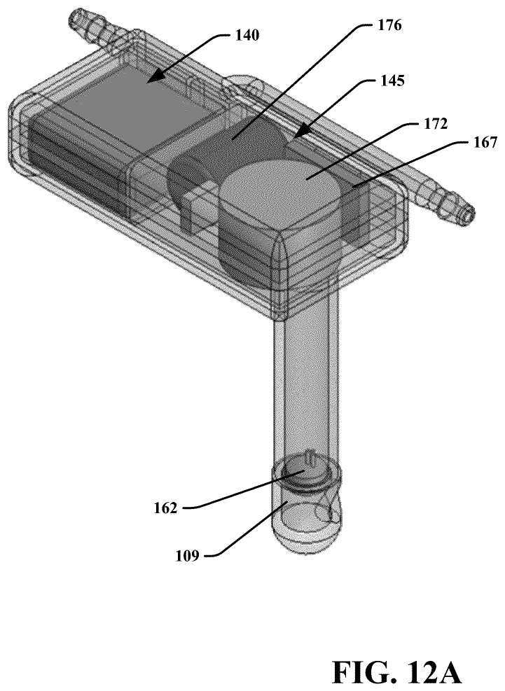

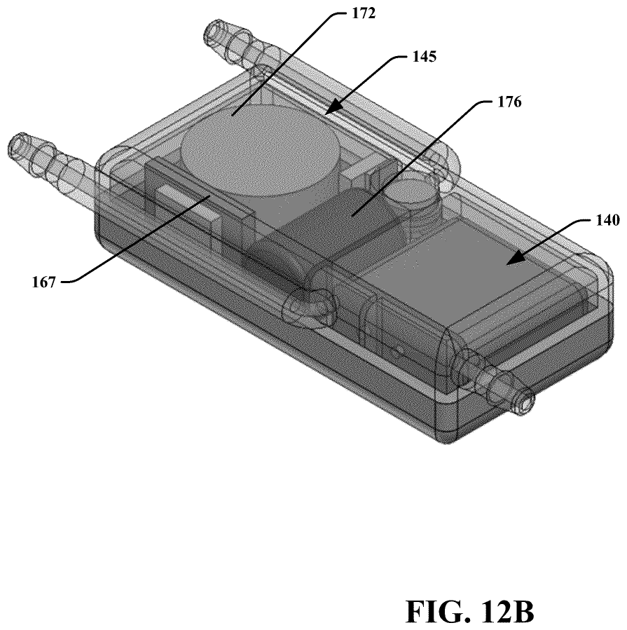

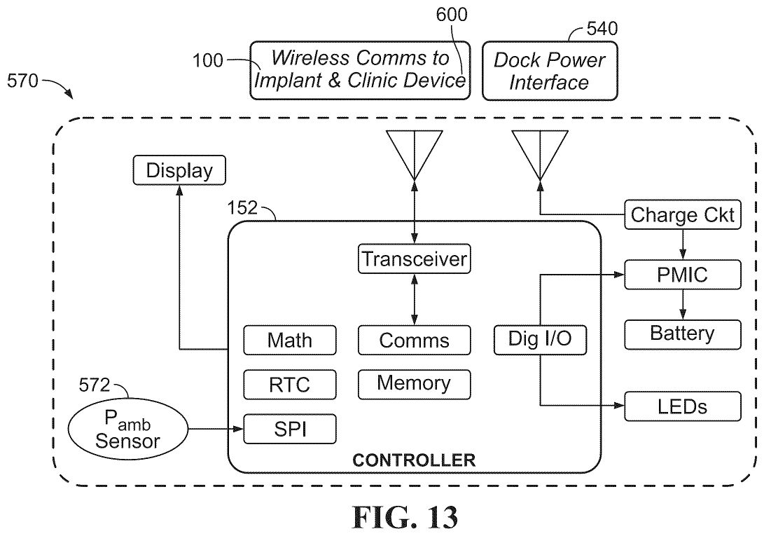

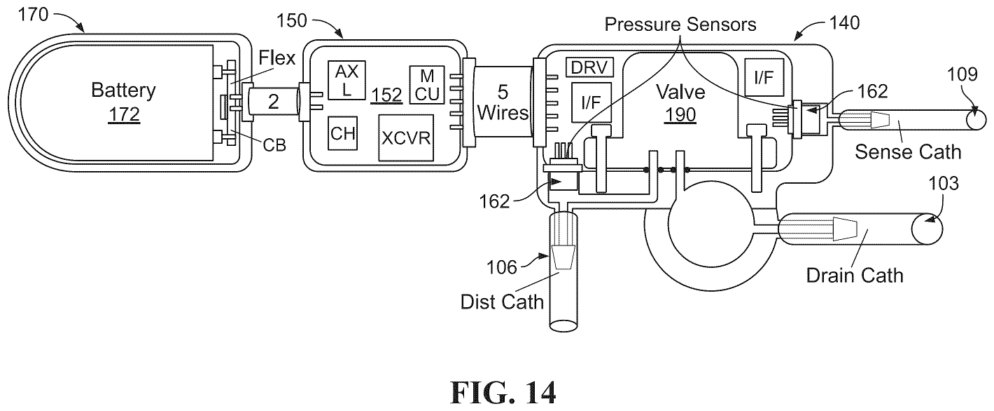

In some embodiments, the pressure sensor 162 itself may be oriented 90 degrees with respect to the axis of the rigid tube 109, with a small window in the side of the rigid tube 109 to expose the pressure sensor's 162 sensitive surface to the surrounding CSF, and preventing excessive force on the pressure sensor 162 when the rigid tube 109 is plunged into the folds of the brain tissue. FIGS. 5 and 10 show a rigid tube 109 without a window (but having a aperture at the distal tip of the rigid tube 109). FIG. 12A shows rigid tube 109 with a window.

Referring to FIGS. 5B-5D, another example of a valve assembly 240 is shown. The valve assembly 280 comprises a valve housing 241 with a tethered capsule 280 connected thereto. The valve housing 241 comprises an inlet 242 for connecting with a proximal catheter 203, and an outlet 244 for connecting with a distal catheter 206. In the embodiment shown, the inlet 242 and the outlet 244 embody barbed fittings for respectively engaging with ends of the proximal catheter 203 and the distal catheter 206. Although it is contemplated that other forms of connection may be utilized, for instance, catheter connectors (e.g., luer lock connectors, friction fit, bayonet connection, etc.), or any other example of a catheter connection disclosed herein or known may be utilized without departing from the present teachings.

A flexible electronic cable 270 may exit the valve housing 241 (via a sealed aperture or connection port 243) and may be connected to the tethered capsule 280 (via a sealed, permanent connection 245). Preferably, the sealed connections (between the electronic cable 270 and the valve housing 241 and the capsule 280) are waterproof and may be hermetically sealed. In the illustrated example, the electronic cable 270 may exit the valve housing at a location adjacent to the inlet 242. It is contemplated that the electronic cable 270 may exit the valve housing 241 in different locations without departing from the scope of the present disclosure, for example, on a side of the valve housing 241 adjacent to the outlet 244 thereof.

A first end 270a of the electronic cable 270 may be operatively connected to an electronics box 245 in the valve housing 241, and a second end 270b of the electronic cable 270 may be operatively connected to the capsule 280. In this embodiment, the electronic cable 270 is employed to transmit information (e.g., a measured ICP) to the electronics box 245 from a pressure sensor 282 in the capsule 280. The use of a flexible electronic cable 270, as opposed to a rigid structure (e.g., a rigid rod), enables the pressure sensor 282 and/or the capsule 280 to be arranged at a variety of different locations in the brain tissue, i.e., in the parenchyma. This aspect of the present disclosure is particularly beneficial for enabling a physician to choose from a wider variety of locations for forming the second burr hole (FIG. 5D). That is, because the electronic cable 270 is flexible, it enables the physician to choose from a greater number of second burr hole locations relative to the valve assembly 240. This may be beneficial, for example, to allow the physician to select a burr hole location in the skull that will not put the patient at risk. In this manner, the valve assembly 240 (via the tethered capsule) gives the physician more freedom, while enabling in-brain measurement with no fluid column.

In some embodiments, the length of the electronic cable 270 may be any length, preferably between about 10 and 60 mm and more preferably between about 20 and 50 mm. In some embodiments, it is contemplated that the capsule 280 may be arranged at a corresponding depth, preferably between about 5 and 20 mm in the brain tissue (i.e., below the skull surface at its closest point). In the illustrated example, the capsule is substantially rectangular. It is also contemplated that the capsule may take on different shapes and forms, for example, a cylindrical-shaped capsule, a circular shape, an ovular shape or any other appropriate shape. It is contemplated that the capsule 280 may have a length of about 6 mm and a width (or diameter) of about 6 mm.

In some embodiments, it is contemplated that the electronic cable 270 and the proximal catheter 203 may be extended through a single burr hole, for instance, the first burr hole shown in FIG. 5D. This may obviate the need for a second burr hole. In some embodiments, an adapter plate, having guide holes provide therein, can be employed and disposed in, over, or near the burr hole with guide holes for receiving and restraining the proximal catheter 203 and/or the electronic cable 270.

As shown in FIG. 5B, the capsule 280 defines a window 281 for enabling CSF to enter the capsule 280, thereby exposing the pressure sensor's 282 sensitive surface to the surrounding CSF. Beneficially, the use of a window 281 prevents excessive force on the pressure sensor 282 when the capsule 280 is plunged into the folds of the brain tissue. In some embodiments, the capsule 280 may be made of a biocompatible metal. However it is contemplated that it may also be made from other biocompatible materials suitable for exposure to brain tissue. In some embodiments, the electronic cable 270 may include connectors on opposing ends thereof, each configured to engage with a connection port on the other of the valve housing 241 and the capsule 280.

Measuring ICP at a location outside the fluid channel can be relevant to the smartshunt system's 1000 automated obstruction detection feature. Turning back to FIG. 5A, if the valve assembly 140, and valve 190 therein, is open and ICP remains high, an algorithm determines that the proximal catheter 103 may be clogged and sends an alert to the patient via the wearable device 570. This alert may notify the patient to contact his/her physician, ideally before the onset of symptoms and possible brain damage.

The electronics box 145 may be made of biocompatible titanium, thin enough to allow RF communications but thick enough to provide mechanical strength and provide hermetic, gas-tight enclosure of the electronics. This configuration will help protect the smartshunt system 1000 from failures due to corrosion and dendritic growth over 10 years of operation in the high humidity subcutaneous environment. The valve assembly 140 may not be a corrosion or dendrite risk as it has no voltage on it for the great majority of its duty cycle (50 hours total over 10 years), and so liquid-proof coating of its contacts and wires may be sufficient. The two rigid boxes for the valve assembly 140 and electronics box 145 may be placed on a thin but strong flexible sheet of Polytetrafluoroethylene (“PTFE”) plastic and connected by flexible fluid or wire tubes. This allows the rigid boxes to move slightly as the patient grows and skull curvature decreases.

The smartshunt system 1000 may comprise two RF antennae 503, 506 (although more than two such antennae may be utilized without departing from the present teachings, e.g., three, four, five, six or more). A small (9×3 mm) communications antenna 503 may reside on a printed circuit board (PCB) 154 inside the electronics box 145. The larger charging antenna 506 is a single loop track that encircles the perimeter of the valve assembly 140. The antennas 503, 506 may interact with antenna 509 of the charger 510 to provide charging of the implant 100. Both antenna 503, 506 operate at well-spaced frequencies so the charging and communications functions may take place simultaneously for uninterrupted operation of the smartshunt system 1000 during charging, e.g., with charger 510.

The charging frequency may be selected so that it and any subharmonics are outside the range of human hearing, while still transmitting through human tissue with minimal loss. This will help avoid annoying the patient with an audible whining sound from the implant 100 during charging. Charging may be based on near field inductive coupling or any other appropriate configuration thereof.

In some embodiments, small barbed fittings may be included on the implant 100 in any appropriate location. By way of example, the small barbed fittings may allow the surgeon to connect the catheters to the valve inlet and outlet. The valve outlet fitting may also provide a small one-way valve, for example a duckbill valve, to prevent backflow into the brain. The total valve assembly 140 may measure 54×20 mm in area, and extends 8.5 mm above the surface of the skull. For comparison, other known devices have the following dimensions 47×16×7 mm.

The embodiment in FIG. 5A comprises the electronics box 145 and the valve box 140 mounted on a flexible base 149. The flexible base 149 may help keep the rigid components from moving away from one another, but be flexible enough to accommodate change in skull shape in size due to patient growth.

Additional embodiments of a smartshunt system 1000 and implant 100 according the present teachings are described below. In the descriptions, all of the details and components may not be fully described or shown. Rather, the features or components are described and, in some instances, differences with the above-described embodiments may be pointed out. Moreover, it should be appreciated that these other embodiments may include elements or components utilized in the above-described embodiments although not shown or described. Thus, the descriptions of these other embodiments are merely exemplary and not all-inclusive nor exclusive. Moreover, it should be appreciated that the features, components, elements and functionalities of the various embodiments may be combined or altered to achieve a desired removable safety chain tie down apparatus without departing from the spirit and scope of the present teachings.

### Implant Alternative Embodiments

The implant 100 embodiment architecture presented above requires two burr holes to be drilled through the skull surface, one for the proximal catheter and the other for the rigid rod containing the pressure sensor. In all versions, the fluid connectors on the valve assembly may be barbed fittings, quick-disconnect, or any other type of fluid tube fitting.

In an alternative embodiment, which may be referred to as dual lumen proximal catheter, an alternative architecture uses a unique proximal catheter. The proximal catheter of this embodiment may contain at least two internal lumens-one for draining CSF and the other for sensing pressure (such as ICP). This embodiment's valve assembly may not include a perpendicular sensor cylinder, as its pressure sensor may be contained within the valve module. Its implantation procedure is essentially the same as for the embodiment described above, with the following changes:

Only one burr hole may need to be drilled into the skull, to allow placement of the dual-lumen proximal catheter. The proximal catheter may comprise an open tip with a tapered feature that allows access by a ventriculoscope such as the NeuroPen by Clarus Medical. There may be no sensor cylinder and no second burr hole that needs to be considered when placing the valve assembly in its scalp pocket. Further, cutting the proximal catheter to length may require cutting each of two separate flexible tubes on the distal end of the proximal catheter. Each of the two separate flexible tubes connects to a dedicated fluid fitting on the valve assembly.

As seen in in the drawings, the two lumens may be joined together starting at the proximal end of the catheter. Both have holes in the portion of the catheter that resides in the ventricle, to allow passage of CSF into the lumens. The two lumens remain attached along the portion of the catheter that resides inside the cranium. Further to distal, shortly after the catheter passes out of the burr hole, the two lumens separate, each going to a separate port on the valve assembly. The drainage lumen, which allows CSF to flow out of the ventricle when the valve is open, functions as in an ordinary proximal catheter and connects to a port on the valve assembly that leads to the valve inlet. The sense lumen, which is flushed with saline or other incompressible fluid during implantation, connects to a separate port on the valve assembly that leads to the pressure sensor. Unlike the drainage lumen, CSF does not flow through the sense lumen. The fluid column is only there to communicate CSF pressure from the drainage holes in the sense lumen to the pressure sensor. Because there is no flow in the sense lumen, it is far less likely to become clogged than the drainage lumen. In this way, the “clog detection” feature of the smartshunt system is preserved. By measuring pressure at a point outside the flow path of the drainage lumen, true ICP will still be measured even when the drainage lumen is obstructed. If the processor detects ICP increase while the valve is open, it can issue a “possible obstruction” alert to the user via the wearable (or external device or external communication device). The drainage lumen may be of larger size than the sense lumen, as only a very small fluid column is needed for pressure communication, but more drainage is generally desired when the valve is open. The barbed fittings on the valve assembly that connect to the two lumens may be sized or shaped differently to prevent mismatch.

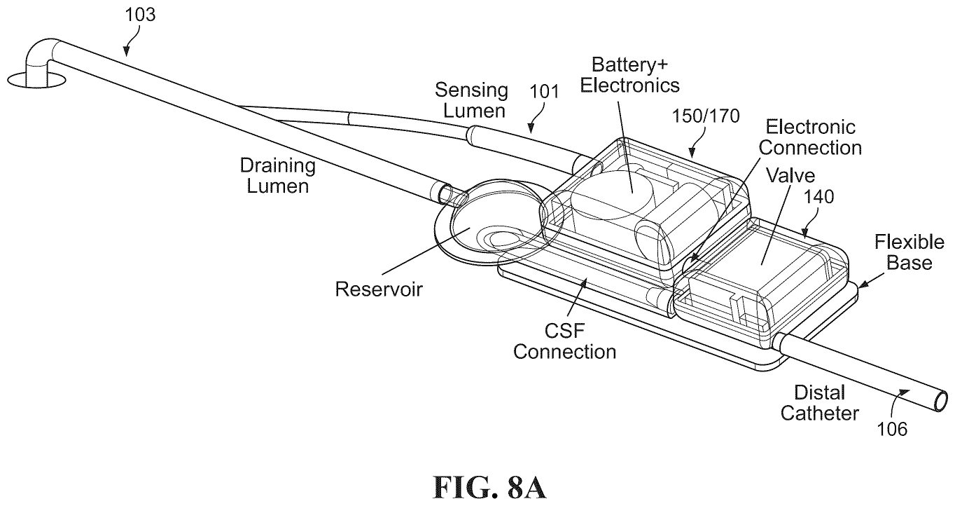

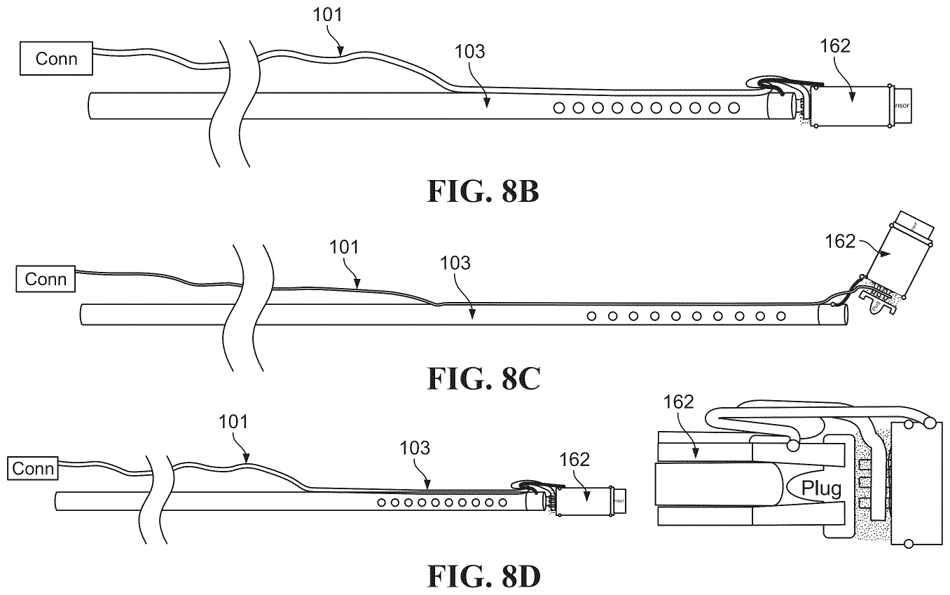

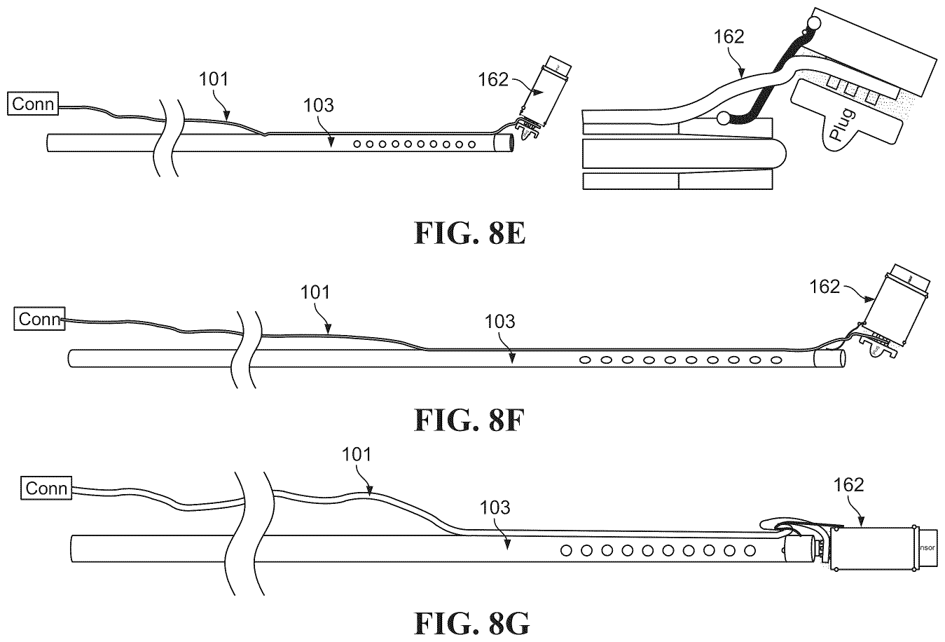

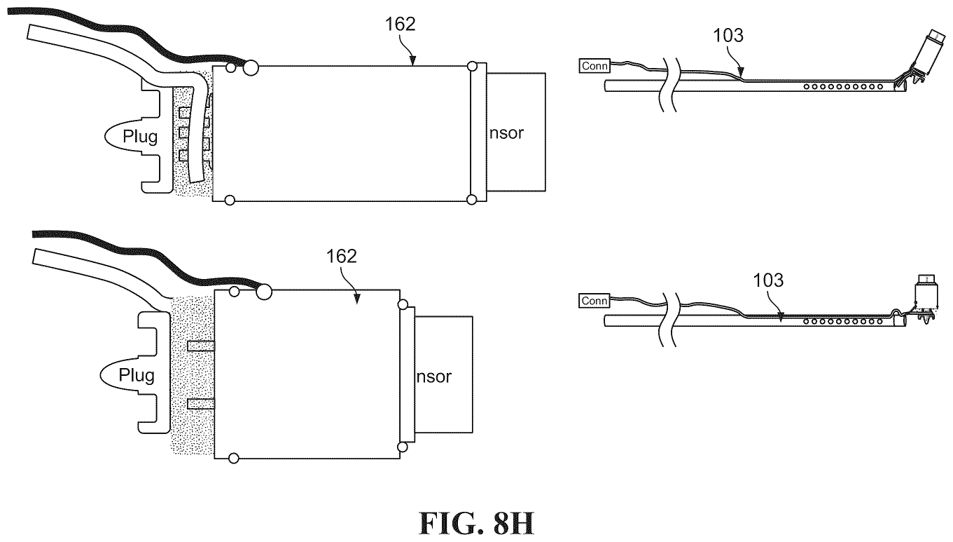

The two lumens may be kept together in the intracranial (proximal) portion of the catheter to minimize the size of the object in the ventricle, as well as to minimize the tunnel diameter the surgeon must create in the parenchyma and the burr hole diameter. They separate distal to the cranium to allow the surgeon to trim each one to length as desired during surgery. An alternative design keeps both lumens together along the entire length of the catheter and has them connect to a custom-shaped port, such as a FIG. 8 shape, on the valve assembly, possibly simplifying the implantation procedure.

This approach has the advantage of direct ventricular, rather than parenchymal measurement. Scarring in the parenchyma may grow onto the sensor's sensitive surface, causing inaccuracy. Likewise, should brain tissue press directly onto the sensor's sensitive surface due to posture change or invagination into the tube, inaccuracy could result. Finally, long-term effects of titanium in the brain parenchyma are not well understood.

The pressure sensor in the smartshunt system 1000 may have sensitivity of 10 μV/V/mmHg, giving only 30 μV/mmHg for a 3V system. The ˜12 cm long wire will go through the CSF and will be sensitive to small changes in resistance in the wire and EMI (both for bridge input power and bridge voltage output). Therefore, signal conditioning at the sensor may be required.

Two architectures for a hermetic capsule that can be placed at the proximal tip of the catheter are proposed. Both use the Renesas ZSC31050 and the Millar TiSense. One positions the Renesas parallel to catheter axis, and the other perpendicular to it. In such embodiments, the proximal catheter may have an outer diameter ≤2.5 mm, inner diameter ≥1.3 mm. The proximal catheter may allow a ventriculoscope (e.g. NeuroPen by Clarus Medical, OD 1.1 mm) to pass through it. The present proximal catheter may be rigid during insertion (e.g. stylet).

The pressure sensor may be on proximal tip of the catheter. The pressure sensor's sensing surface must face out of the catheter to enable clog detection. The proximal catheter must be safely removable.

In such embodiments, the implant's 100 lifetime shall be greater than 5 years (shall), and potentially even greater than 10 years. The proximal catheter length may be adjustable between 4.5 and 11 cm.

A flexible fluid tube may be 110 mm long and may comprise drainage holes at proximal (right) end. A cylindrical sensor capsule, 14×φ4 mm, may non-permanently be plugged into the proximal end. An electrical cable (yellow line) may extend from the sensor capsule to attach to the fluid tube near a proximal end. A cable 35 mm distal, may detach from the fluid tube. This may allow the surgeon to cut the fluid tube to a desired length, such as between about 40-110 mm (within 1.5 mm). The sensor capsule may be tethered to the fluid tube with a metal tether. The cable may terminate distally in a small electrical connector that will connect to the valve assembly. The smartshunt system may be shipped and inserted into brain with sensor capsule plugged into the fluid tube.—FIG. 8B.

After positioning in the ventricle, the surgeon may remove the stylet used to place the catheter and may insert a slightly narrower stylet designed to push the sensor capsule out of the fluid tube. The tether holds the sensor capsule in place and it remains in this configuration throughout the device lifetime. See FIG. 8C.

As shown in FIG. 8D, a 1.4 mm OD stylet may push tapered end cylinder but does not touch plug during ingress through the brain to the ventricle.

After placement in ventricle, the surgeon/clinician may remove the 1.4 mm stylet. Then the surgeon/clinician may insert 1.1 mm OD stylet to dislodge the sensor—see FIG. 8E.

The surgeon/clinician may remove the 1.1 mm stylet. The path is now clear for 1.1 mm OD NeuroPen ventriculoscope, if desired. Further in some embodiments, the inner lumen is 1.5 mm. Taper in endcap goes from 1.5 down to 1.2 mm and the NeuroPen is 1.1 mm.

The sensor capsule may remain tethered near the open end of the catheter in normal operation. Should the proximal catheter needs to be explanted, the surgeon pulls the fluid tube and cable together. The sensor capsule will naturally stay in line with the catheter as it is pulled through the parenchyna tunnel and burr hole—see FIGS. 8F and 8G.

Additional embodiments may include concepts where the pressure sensor is mounted on the tip of the proximal catheter. To allow vision with the ventriculoscope, such a version may mount the sensor off to the side of the main lumen, or may divert the scope's lumen to be parallel (or generally parallel, i.e., within 3-5 degrees thereof) to the sensor at the tip. Yet another embodiment may feature a capsule at the proximal tip containing the pressure sensor, but with a 45 degree turning mirror on the distal side of the capsule. This may direct light through a hole in the side surface of the catheter. The ventriculoscope could then ‘see’ through this hole and off of the turning mirror, to obtain a view perpendicular or generally perpendicular to the longitudinal axis of the catheter.

In another embodiment, the system may comprise two separate single lumen catheters. In yet another alternative architecture, the valve assembly may be configured as in the single burr hole version described above, with separate fluid ports for sense and drain channels. But, instead of using the dual lumen proximal catheter described above, the surgeon may use two single-lumen proximal catheters. The catheters may be placed in separate burr holes, or one large burr hole. The catheters may be placed in separate locations in the brain, or in the same location. For example, one might place the sense catheter inside the upper parenchyma of the brain, just below the subarachnoid space, and the drain catheter in one of the ventricles. Each catheter connects separately to its appropriate valve module/assembly port such as described above.

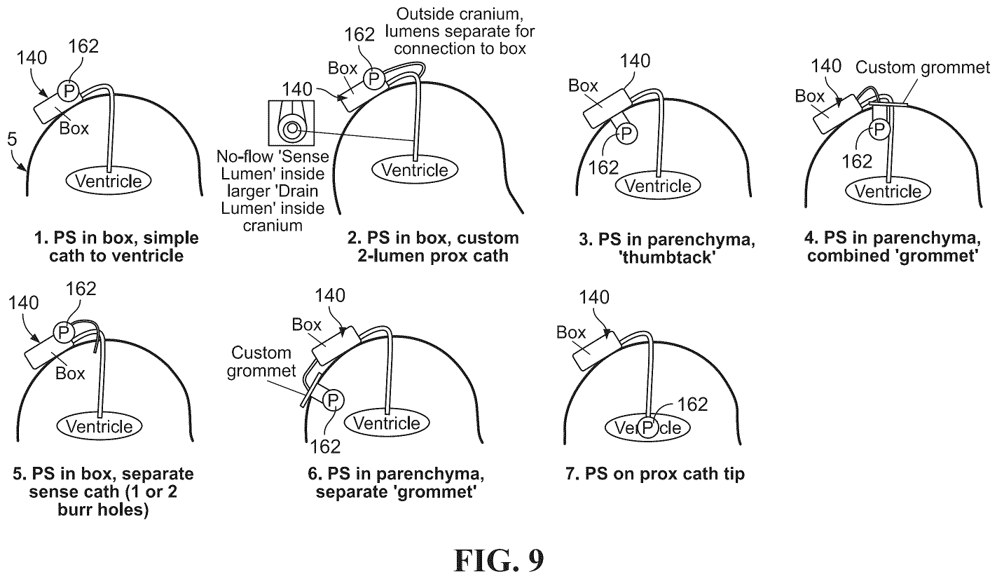

A further alternative may comprise a sensor in separate housing connected by wires. Some embodiments, where the sensor is located outside the valve module/assembly may have free electrical wires from the sensor that must be connected to at least one electrical connector on the valve module. In these cases the implantation procedure is the same but with the additional step of making the electrical connection to the valve module. This may include cutting or stripping the wires, or may be configured for insulation penetrating connections, screw terminals, or other means known in the art for the surgeon to carry out the electrical connection. The surgeon may pot the electrical connection with an insulative coating. One embodiment of this alternative may place the ICP sensor on the proximal tip of the proximal catheter (configuration 7 in FIG. 9).

In yet another embodiment, the system may comprise a plate with holes covering single burr hole. In all versions, a dedicated plate may be used to cover the burr hole and facilitate catheter placement, whether one, two, or more catheters are used, as shown in FIG. 9 (configuration 4). In such embodiments a ninety degree fluid fitting may also be used over the burr hole to minimize catheter bend radius. In such embodiments, additional separate devices, such as fluid reservoirs, Rickham reservoirs, or anti-siphon devices as are known in the art may be placed in series or parallel with the valve module.

In combination with, or separate from, the above embodiments, numerous other Implant variations and alternatives are possible. Some of these are listed below:

Many types of electromechanical valves may be used besides those based on shape memory alloy (SMA). These may comprise: solenoid valves; pinch valves in which the actuator pinches a tube closed to reduce flow; piezoelectric valves; motor drive valves, or any other electromagnetic (“EM”) valves that are of small size, low power, and quiet operation. Although a latching valve is disclosed in the some of the embodiments, non-latching valves may be used as long as power requirements are met, which depends on the valve's holding current requirement as well as the amount of time the valve must be held in a given time period. For latching valves, both magnetic and mechanical latches may be used.

The valve should switch quietly, with no audible click that could annoy the patient. SMA valves are generally quieter than solenoid valves. Slow switching transitions, or sound dampening material placed around the valve may also be used to control noise.

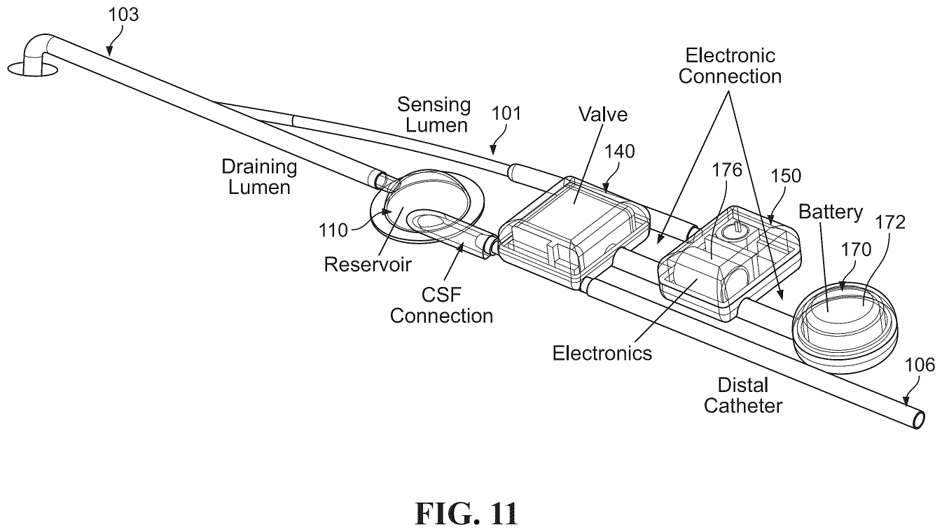

The size, shape and location of the components: reservoir, valve box, and electronics box, can vary for both the ‘rigid tube’ and ‘double lumen’ approaches—see FIG. 5A, FIG. 8A, which depict the rigid ‘box’ components as residing on a flexible base, but other layouts are possible, such as those shown in FIGS. 10 and 11. In one embodiment, both the valve box and electronics box (and possibly the reservoir) are all located in a single box as depicted in FIGS. 12A-B. The box may have an interior wall or double-wall that separates the gas-tight, hermetically sealed volume that contains the electronics from the non-hermetic volume containing the valve or the reservoir. The single box may have a curved or ridged bottom to accommodate skull curvature. The physician may create a recess in the skull to accept the box.

Another component layout does not have a flexible base, but connects the various rigid components with flexible fluid or electrical connections, as in FIG. 10 and FIG. 11. This may have the advantage of better accommodating skull size and shape changes as the patient grows.

The Implant may include a second pressure sensor for measurement downstream (distal to) the valve, which measures intra-abdominal hypertension (“IAH”). This could eliminate the need for a wearable in some embodiments.

Further, the valve assembly, or parts of it, may be located inside the patient's thorax, abdomen or some other ex-cranial region that allows for larger sized components.

Bluetooth Low Energy (BLE) may be utilized in the described embodiments as a wireless communications protocol. But other protocols are possible, including: peer-to-peer protocols such as Bluetooth Classic or NFC; many-to-many protocols such as Bluetooth Low Energy, Bluetooth 5, WiFi Direct, NFC, Zigbee, Z-Wave and 6LoWPAN; Local Area Network protocols such as WiFi, cellular long-range protocols such as GSM/GPRS, 4G, 5G, or LTE; and Low Power Wide Area Network (LPWAN) protocols such as LoRa, LoRaWAN, LTE-M, or NB-IoT, as well as custom wireless network protocols.

Lithium Ion battery chemistry is contemplated in the main embodiment, but other high-density energy storage technologies are possible, including Lithium Iron, Lithium Polymer, Nickel Metal Hydride and others. Also, a primary cell or battery may be used instead of rechargeable secondary cell, if very long lifetime is not required.

The main embodiment description includes a relatively large supercapacitor in the electronics box that provides peak current for valve switching and is trickle-charged by the battery in between valve transitions. Alternatively, a battery with larger peak current capability may be used, which would obviate the need for a supercapacitor or capacitor to provide peak current.

Instead of titanium, the gas tight hermetic box may be built from any gas-tight material, including other metals or ceramics, including glass. If the housing is conductive, a ceramic or glass window may be welded to it to prevent electromagnetic shielding of the wireless charging or communications signals.

For the dual-lumen architectures, the two lumens may remain combined together and not separate outside the skull for separate connection. Instead, the surgeon may just cut the single dual-lumen catheter to length and attach it to a single barbed or other type of fluid fitting on the valve assembly. The attachment point may be keyed to prevent improper mating, and each lumen may attach directly to a hard tube on the valve assembly that fluidly couples it to either the sensor or the valve.

For the dual-lumen architectures, there could be a specialized or unique fluidic fitting on the valve assembly to prevent a surgeon from mistakenly using a standard third party proximal catheter on either the drain or the sensor port and neglecting the other port.

In medicine, ICP is universally described as a gauge pressure, that is, the cranial pressure relative to the ambient air pressure in the patient's surrounding environment. ICP measurement devices, extraventricular drains (EVDs), medical guidelines and medical literature all express ICP as a gauge pressure. Since ambient pressure (Pamb) is constantly changing due to weather conditions or patient movement to different altitudes, accurate gauge ICP measurement requires measurement of both the ICP and Pamb. However, because the implant is fully inside the body, it has no means to measure Pamb. Conventional VP shunt valves do not actually operate on gauge pressure, but on a differential pressure between the proximal and distal sides of their valves, making them susceptible to inaccurate pressure switching when abdominal pressure or posture changes. External ICP Monitors and EVDs have direct access to both in-body and ambient pressure, and so can deliver true gauge ICP measurements.

The present smartshunt system overcomes the inaccuracy due to Pamb changes by means of an external communication device (also referred to as an external device) that may comprise a wearable device (such as that described above). The wearable device may comprise a small, battery-powered, wireless device that measures Pamb frequently and communicates it to the implant via the BLE wireless link. In these embodiments, the patient keeps the wearable within a specified distance, typically three meters, of their person. Physically, the wearable is a small electronics box approximately 30×30×8 mm in volume. After shunt implantation, the patient will be issued two wearables, one to be docked for recharging while the other is worn. The wearable housing should be attractive, rugged, comfortable, and unobtrusive, for example in the form of wristwatches/bracelets, necklaces, pocket clips, ring, pin, clip for a belt buckle and possibly a smartphone app. Further still, while the term wearable is utilized, the external communication device may also comprise a device that is able to be held by the patient, such as in his/her pocket (any pocket would suffice), such as a separate device, a pocket watch, a puck-shaped device, a clip-on or the like. The external communication device may also be integrated into a separate device that could provide a dual use. It may also be integrated into a piece of clothing for the patient, e.g., a shirt, hat, t-shirt, vest sweatshirt or the like. The external communication device is configured to measure Pamb and communicate same to a third party device, the implant, a microcontroller in the implant or the like.

Should a wearable become nonfunctional due to loss, damage, or internal failure, the patient may receive a notification on a separate communication device, such as a smartphone or computer. Further still, the patient's physician or other related third party (such as a parent or guardian) may receive a notification in addition to or in the alternative to the patient.

The drawings contemplate a block diagram of key wearable components for some embodiments of the present disclosure. The wearable may comprise a transceiver and processor chipset, which may be the same as for the implant. This may allow BLE communications between the wearable and implant, as well as between the wearable and the device hosting the clinic app (“clinic device”), as detailed below. The wearable may also comprise a Pamb sensor. At left, the Pamb sensor is a high accuracy air pressure sensor with built-in temperature compensation. The small display is a simple alphanumeric display using low power E-Ink or other “E-paper” and is used to provide the wearer with the on-demand ICP and device status reading. On the right of the figure, the power management circuit allows wireless recharge of the wearable when connected wirelessly to the dock, with LED's providing charge progress status.

Besides providing Pamb information to the implant, the wearable also wirelessly receives the data from each implant measurement. It checks for transmission errors, adds a real-time timestamp, and logs the reading in its memory for future download by a physician. Each reading may comprises:
Absolute ICP (moving window average over 10 seconds)
Patient posture relative to gravity (moving window average over 10 seconds)
Pamb (as measured by the wearable)
Implant serial number
Implant valve state (after reading)
Implant valve settings: open threshold, close threshold, valve state on power loss, closed state measurement interval, open state measurement interval, ICP rolling average window width (in seconds)
Estimated time to next implant recharge
Any alerts: low battery, suspected catheter clog, missed readings, ‘contact physician’ system errors

In some embodiments, the implant may take a reading every 15 minutes when its valve is in the closed state, and every 30 seconds when in the open state, in order to prevent overdrainage. At any time, the user may display the ICP from the last reading, as well as time of the last reading, valve state, time until the next reading, and any alerts by means of a pushbutton on the wearable.

Further, in some embodiments, the wearable may serve as the interface to the clinic app during office visits. The wearable may pair with the third party Bluetooth device (typically a Windows tablet or laptop) being used by the physician. It carries out the following functions when requested by the physician using the clinic app:
Datalog upload. the wearable uploads its datalog, which contains 180 days worth of data (if after this time wearable memory becomes full, it deletes oldest data first). The clinic app also provides graphic and other analytical tools for convenient storage, search, filtering, post-processing and display of longitudinal ICP data for all enrolled patients. After successful data upload, the wearable deletes the uploaded data to free up memory for future readings.
Settings Change. The physician opens a screen on the clinic app that displays all current settings for the implant. The physician changes settings on the screen as desired and issues a “save” command. The clinic app writes the new settings to the wearable along with a timestamp. The next time that wearable communicates with its implant (based on the implant's sleep cycle and typically within 15 minutes), it writes the new settings to the implant.
Re-Calibration. Initial system calibration for posture (tilt) offset takes place on the first day after recovery from implantation surgery. As the patient grows, annual re-calibration at an office visit may be carried out to determine the offsets due to posture that must be programmed into the implant's memory. This ˜10 minute office procedure involves taking ICP readings with the patient sitting upright, then laying down in the prone, supine, left decubitus and right decubitus positions. The software writes the new offsets into the wearable memory for each position and the wearable communicates these to the implant at the next communication session.

For optimal RF communication, the wearable's housing may be of a nonconductive material such as plastic or ceramic. The housing should be sufficiently gas permeable for the internal pressure sensor to accurately measure ambient pressure. This may be accomplished by including vent holes in the wearable's housing, possibly with a water resistant screen. The wearable must be worn (or in close proximity to the patient) at all times including outdoors in all weather conditions. This requires it to operate in all outdoor temperatures and humidity levels, as well as to be moisture resistant in rainy conditions. Instructions For Use (IFU) may require the patient to wear the wearable (or be in close proximity thereto) under an inner layer of clothing in cold conditions, as these may impact battery operation, as well as in rainy conditions to prevent moisture damage to the electronics. In some embodiments, the wearable may be waterproof, suitable for use while swimming or in rainy conditions.

The wearable may also feature a “find my device” tag to allow a lost wearable to be tracked by a phone app. As hydrocephalus patients come from all age, gender, and cultural backgrounds, a variety of external housings may need to be developed. These can include necklaces or wristwatches of a variety of colors, designs, or graphics. As some patients may wish to conceal their wearable, non-visible clips or attachments to undergarments are a suitable alternatives. A wearable may also be implemented as a smart phone app, for patients who can be relied on to always keep their phone with them.

The wearable should be rugged and survive frequent drops, while protecting the electronics inside. It should be easily cleanable, typically by wiping with a damp cloth or with a mild solvent such as 75% isopropyl alcohol.

By using a Beacon mode, any wearable can provide Pamb to any implant. However, a given wearable may only upload data from its designated implant, using a serial number handshake.

### Wearable Alternative Embodiments

In other embodiments, the smartshunt system may not include a wearable. Instead, Pamb uses IAP as surrogate. The wearable described in the above embodiment provides the implant with the current ambient pressure (Pamb), allowing the implant to convert its absolute ICP pressure measurement to ICP gauge by subtracting Pamb. This system has the advantage of using accurate Pamb. However, it requires the patient to always keep the wearable within several meters of their body. An alternative architecture eliminates the need for a wearable by using another in-body pressure, for example peritoneal pressure, as a surrogate for Pamb. Peritoneal pressure generally tracks Pamb in real time, usually adding only a small amount of pressure to Pamb due to gas, ascites, muscle flexure, fluid column weight, or other causes. The term ‘Intra Abdominal Pressure (IAP)’ is often used to describe pressures in the abdominal area, even though these can vary somewhat between organs and locations. For the purposes of this disclosure, use of IAP to generally represent any region in the abdomen whose pressure generally remains near to that of Pamb, including within the peritoneal sack.

To obtain Pamb by measuring absolute IAP, a second absolute pressure sensor is placed in the implant's valve assembly, this one measuring pressure downstream (distal to) the valve, near the point where the distal catheter connects to the valve assembly. An example layouts are shown in the various drawings. This IAP sensor's absolute reading will be the IAP plus the weight of the fluid column in the distal catheter PFC, plus any offset pressure added by the body PBO. To obtain Pamb, the following formula may be used:

$$Pamb = IAPa - \{ PFC + PBO \}$$

where IAPa=absolute measurement from the IAP sensor; PFC=fluid column weight in the distal catheter, and; PBO=offset caused by the body due to gas, flexure, etc.

To determine the value of {PFC+PBO}, a one-time, noninvasive measurement may be taken during an in-patient office to derive calibration coefficients that are written into memory. An external unit (or ExU, a device similar to the wearable but not required to always be with the patient) may be used to interface the physician's laptop or tablet device to the implant.

First, the patient sits upright. The ExU commands the implant to close the valve so that the IAP sensor is not influenced by ICP. Then it takes an absolute IAPa measurement and uploads it to the ExU, which measures the current Pamb and subtracts it from IAPa to obtain gauge IAPgU. The patient then lays fully recumbent, for example supine, and a second absolute IAPa measurement is taken, uploaded, and current Pamb subtracted to obtain PBO. The ExU computes a calibration value:

$$PFCU = IAPgU - PBO$$

where PFCU=pressure due only to fluid column when the patient is fully upright, and IAPgU is the {IAP gauge pressure plus Body Offset pressure} that was measured when the patient is fully upright.

Both PBO and PFCU are transmitted to the Implant and written to memory, and are used in every future pressure measurement, until a recalibration occurs. PBO, the pressure offset caused by the body, is assumed to be constant for every measurement. PFCU must be multiplied by the cosine of the angle θ that the patient's thorax makes with gravity, as measured by the tilt sensor in the Implant. Substituting values:

$$Pamb = IAPa - PFCU cos θ - PBO$$

where IAPa=absolute measurement from the IAP sensor; PFCU=fluid column weight in the distal catheter measured when the patient is fully upright; θ is the angle the patient's thorax makes with gravity as measured by the tilt sensor, and; PBO=offset caused by the body due to gas, flexure, etc. The patient should be re-calibrated periodically during office visits to account for growth or changes in the PFCU and PBO. As a pediatric patient grows taller, the distal fluid column lengthens and PFCU increases. Existing anti-siphon devices (ASDs) switch on a fixed value which changes as growth occurs. The Smartshunt system's annual calibration ensures that the PFCU value subtracted is optimized for the patient's current fluid column length.

To account for fluid column weight in the distal catheter, θ should be the angle the distal catheter makes with gravity, with zero indicating the thorax is parallel to gravity (patient upright). If the tilt sensor is placed in the valve assembly, which is generally located in the patient's head, the smartshunt system may measure an erroneous θ when the head is tilted at a different angle than the thorax. This can be overcome by placing the tilt sensor in the thorax, below the lowest vertebra of the neck. This may be accomplished with a wire attachment to the valve assembly, or by placing the entire valve assembly below the neck.

In order to account for both the fluid column in the proximal as well as the distal catheter, two tilt sensors may be required-one for the thorax and the other for the cranium, in order to account for all possible combinations of head and thorax tilt. In this embodiment, one would need to measure separate upright and recumbent fluid column pressures for the head and thorax, using the upstream and the downstream pressure sensors, respectively, each time with the valve closed. These calibration offsets may be applied to the upstream (proximal, for ICP) and downstream (distal, for IAP) pressure measurements separately for each future reading.

An alternative embodiment to cancel out the influence of the distal fluid column is to place the downstream (distal, IAP) sensor exactly halfway down the length of the distal catheter, balancing the fluid weight above and below the sensor so that the resulting PFC is zero for any θ. In this case, a tilt sensor may not be necessary. The IAP sensor may be attached to the inside of the distal catheter, or externally on a T-piece, with wires running to the valve assembly. Or, the entire valve assembly may be placed at the same location in the thorax as the distal catheter midpoint. The same concept may apply to the smaller fluid column in the proximal catheter.

Rather than rely on a one-time calibration to measure the IAP added by the patient's physiology, the instructions for use could require the patient to take periodic (e.g., daily) readings from the ExU to obtain true Pamb. The two-sensor system could then use machine learning to correct for IAP added by the patient's physiology, as well as tilt, by averaging past readings, either with a weighted or unweighted rolling window.

In further alternative embodiments, other body pressures could be used instead of IAP as the surrogate for ambient. For example, lung or chest cavity pressure at end expiration could be used.

In further embodiments of the smartshunt system, periodic ambient reading from ExU may be utilized. In this alternative embodiment, an “External Unit” (ExU) has essentially the same functions as the wearable, but is not required to be within several meters of the patient at all times. Instead, the patient will be instructed to place the ExU within wireless range (typically several meters) periodically, for example once per day or once every eight hours. The ExU then communicates the current Pamb to the implant, and the implant stores this value and uses it until the next communication. Communication may be automatic, using the ‘beacon’ concept described above or a pairing protocol. Communication may also be triggered by patient action such as a button push on the ExU. Other ExU functions, such as the on-demand ICP reading, datalog download, settings changes, etc., could still be as in the embodiments described above.

This alternative has the advantage of not requiring the patient to wear the ExU at all times, but may cause undesired valve states if Pamb changes between updates due to weather conditions or altitude changes.

Other wearable alternatives and variations-several other possible alternatives to the embodiments above are listed below:

The wearable could be held against the patient's body or head using an implanted and external magnet. The preferred embodiment teaches the use of the same communications chipset in the wearable as in the implant, but obviously different chipsets may be used. The wearable may also use any of the available secondary battery types, or may use a primary battery for a shorter lifetime. The wearable may contain electronics to communicate with the internet via the cell phone, Wifi, WLAN, or other network. With this capability, it could upload data directly to a cloud database, and download new settings from that database. The physician's smart device would then only have to interface with the cloud database. Likewise, the patient would use a phone or computer app to access the database to obtain the latest ICP reading. In this variation, the wearable might not require a display.

The implant may be fitted with a chipset enabling it to connect directly to the internet, eliminating the need for the wearable's data transfer function. In this case the wearable would only serve in beacon mode for Pamb measurement.

In a further embodiment, the cloud server or the implant could determine the implant's GPS coordinates by methods known in the art, and the cloud server would determine Pamb based on GPS coordinates. It would assume the patient is in a commercial aircraft for GPS readings above a certain altitude and make an appropriate assumption for Pamb. In this case, the wearable could be eliminated.

The wearable could also use GPS for location and eliminate its internal pressure sensor. In these embodiments, the wearable may comprise a GPS system and it may be utilized as described above.

The embodiments above mentioned LEDs as a user interface, but other interfaces known in the art can be used, including audible or haptic interfaces may be utilized. Further still, other display systems may be utilized without departing from the present teachings.

In the embodiment descry bed above, data processing functions such as averaging, spike elimination, ambient P subtraction, tilt correction for fluid column, etc., are carried out using the implant's processor. However, such processing can be shared between the wearable and implant in other embodiments. In an example, the implant could stream all raw data samples from its ICP sensor and/or tilt sensor directly to the wearable, and the wearable could perform all averaging, fluid column correction, etc. The wearable could then decide on the desired valve state and simply command the implant to set the valve to that state.

The wearable and implant could also share processing and decision-making functions with one or more third processing devices.

In the embodiments described above, the implant carries out timing to initiate measurement/valve control events and uses the wearable only as a beacon for Pamb. It then changes to a more conventional Bluetooth pairing relationship to upload its data to the wearable. An alternative architecture could have the wearable initiate measurement and communication events rather than the implant.

Throughout this description terms “cell” and “battery” are used interchangeably, even though our implant's power source is a single cell.

Based on our present power draw model, the implant's 46 mAh rechargeable, lithium ion coin cell should be able to power the smartshunt system for at least 14 days given worst case usage assumptions and for 30-40 days with typical usage assumptions. The implant's processor estimates remaining battery life by tracking its “on” time in the various modes, and mathematically determining remaining charge. “Estimated time to next charge” is one of the smartshunt system status parameters the implant uploads to the wearable at the end of each reading. The wearable displays time to next recharge along with the ICP and other data during on-demand readings. If charge is overdue, the wearable will provide an LED alerting the user that an implant recharge is needed.

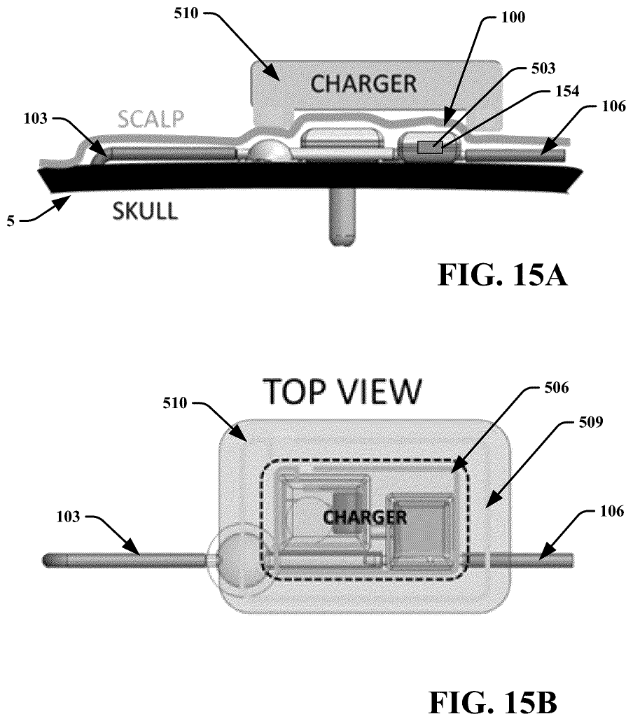

The smartshunt system may comprise a charger of any appropriate configuration. By way of a non-limiting example, the charger may comprise a small, plastic box with dimensions 60×40×12 mm. It may be powered by its own lithium battery. When not in use, it rests on the dock and is recharged there wirelessly. When the patient's wearable indicates that a recharge is due, the patient must hold the charger against her head for a total of 4-6 hours. This may be done in one session or in several shorter sessions over several days. To facilitate charging, a headpiece may be provided to allow free motion and hand use during charging. The patient attaches the charger to the headpiece and fastens the headpiece to her head. The headpiece allows charger placement anywhere on the cranial surface, as patients' valve assemblies may be in different locations. The charger has a recess shaped to mate with the portion of the valve assembly that causes a slight protrusion in the patient's scalp. This makes it easier to locate the valve assembly and to position the charger to ensure optimum alignment of the devices' antennas. The antennae are loops around the perimeter of each device. Energy transfer is accomplished with inductive coupling, and so optimum charging (shortest charge time) requires the charger's antenna to fully encircle that of the implant. In the “top view” of FIG. 15B, the recessed area is represented by the dashed line, and the antennae as solid lines around the perimeters of the implant and charger. The recessed area can be a simple rectangle or any arbitrary shape, for example the recess could be extended so as not to compress the reservoir or the catheters.

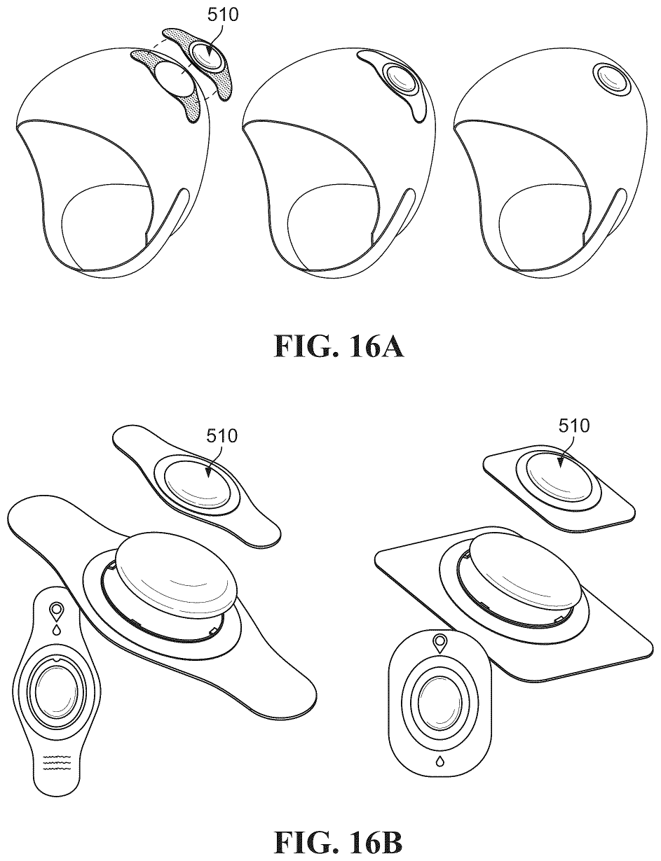

The charger should be easily inserted into and removed from the headpiece, to allow the headpiece to be washed (ordinary washing machine) and the charger to be cleaned (wipe with damp cloth). FIG. 16A illustrates an embodiment of the headpiece and two concepts for the external design of the charger. The two different designs at bottom show the rigid box that contains the charger circuitry as part of a Velcro patch, removable from the patch and with a round recess to fit over the Implant bulge on the head surface. The upper left sketch illustrates a concept for the headpiece, covered with the nap side of the Velcro, allowing the charger patch with the hook side of the Velcro to be placed anywhere on the headpiece. The upper center sketch has the patch permanently attached to the headpiece, but with the charger removable, and the upper right has the charger permanently sewed onto the headpiece. The upper left concept has the advantage of allowing placement anywhere on the skull surface, as the implant may reside anywhere on the skull surface. It may also accommodate movement of the implant over time.

When not in use, the charger rests on the dock and its own battery is recharged. Besides design for power efficiency, the charger's RF frequency for recharge is selected to be outside the range of human hearing, to avoid annoying the patient with a whining sound in the implant or in the docked charger. Power management circuitry in the implant and charger may prevent hazards such as overheating.

The implant's battery starts automatically recharging any time a charger is brought within range of the implant. None of the other implant functions—timed reading, for example—is affected by the presence or absence of the charger. If a patient neglects to charge the implant when prompted, the implant continues to function and issue its alert to the wearable, which provides a visual, audible, or haptic indication to the patient. If the Implant determines that its battery will fail (dip below its minimum specified voltage) soon (within a preset time threshold), it automatically places the valve in its “safe state” and then switches itself off in a safe, controlled manner. This is to prevent random system behavior, which may occur when the battery falls below its specified voltage minimum. The “safe state” of the valve is ‘closed’ by default, but may be ‘open’ for some patients and can be set by the neurosurgeon using the clinic app. The implant will thus stop taking timed readings and contacting the wearable. If a wearable is not docked and does not receive a signal from the Implant within a specified time (for example, 45 minutes or three times the typical timed reading interval), it issues an audible, visual, or haptic “no implant” alert, for example via an LED. The smartshunt system will remain in its powered down state until a Charger is again brought into range of the Implant. The Implant then automatically starts battery recharge. Only when battery voltage and capacity are at a safe level again will the Implant's processor boot itself up and restart the timed reading cycles. The Charger has a means to detect (RF load monitoring) whether it is properly positioned and charging the battery, even if the Implant is powered off. Before auto-shutoff due to near-dead battery, the Implant logs the time of shutoff. It also logs time of bootup after battery power is restored.

The charger may be interchangeable, i.e., any charger can interface with any dock or implant.

The following are several alternative embodiments of the charger device. In one embodiment, the charger and wearable may be combined into a single device. The charger is not portable and does not have its own battery. It is powered by a cable coming from a conversion box that draws power from a home wall outlet. The headpiece has one or more charging antennas built into it, which connect to a remote charger box via a cable. The headpiece has the entire charger built into it in the form of a flex circuit, or a flex circuit combined with rigid components such as a battery. The headpiece is a simple harness, a rigid helmet, or some other form of headgear. There is no headpiece. The charger is built into a pillow that the patient lays his head on to charge. The smartshunt system's power pack my be located away from the head in a subcutaneous location, for example in the thorax. The charger may be placed at that location. The battery is primary and is changed out periodically with a simple surgery, and there is no charger. The charger contains a numerical display or progress bar indicate charging status.

Further, the purpose of the dock is to (1) recharge the wearables' and charger's batteries and (2) provide a standard place to store wearables and charger when not in use, preventing the patient from misplacing them. The dock plugs into a wall outlet at the patient's home and should be placed in a location within 3 meters of the patient's head while the patient is in bed, allowing the docked wearable to recharge and communicate with the implant while the patient sleeps. A dock can simultaneously charge the one charger and two wearables given to each patient after implant surgery. While one wearable is worn, the other is charging on the dock. This ensures that the patient always has at least one fully charged wearable available.

The dock is a flat platform with marked spaces on it for two wearables and a charger. There may be recesses in the dock surface to accept the devices. As it is generally kept on a nightstand, the dock should be silent and dark, with no lights or sounds that could interrupt sleep. The dock should be small and lightweight, and should pack easily for travel.

Other embodiments of the dock may incorporate self-test capability for the devices, and an internet connection to allow upload of wearable data logs and charger health data to a cloud database for physicians to track patient data or for a third party to track device health and maintenance.

Further still, the dock may issue an alert (sound or LED) if it does not have at least one wearable in it. This prompts the patient to always keep at least one wearable charged. There may also be an alert from the dock if no charger has been placed on it within a preset time limit, for example 48 hours.

Likewise, every wearable must be either in regular contact with an implant, or docked. If it is not in one of these states, the wearable may issue an audible and visual “no implant/dock” alert.

The dock may have its own large backup battery to keep charging wearables and charger during power outages to the home. Docks may be interchangeable and may work with any wearable or charger. The dock may be free of a flat surface for wireless charging, but each device may have a simple power supply that plugs into a wall and plugs into the wearable or charger directly. Rather than a larger horizontal surface with multiple marked areas for the devices, the dock could be several smaller, separate docks. Instead of wireless charging, the dock could take the form of a cradle that the devices plug into with a built-in connector or a slot.

The clinic app is a software application that resides on a Windows device (or IOS device) with Bluetooth capability—typically a laptop or tablet. The clinic app may be used only by neurosurgeons in a clinical setting, usually an office visit at a hospital, clinic, or doctor's office. In the future, the clinic app may expand its IFU to allow other types of clinicians—neurologists, physician assistants, nurse practitioners, or nurses for example—to perform some of the less critical functions, for example data download or settings changes if prescribed by a neurologist.

In operation, patients may bring their wearables to an office visit. The clinician uses the clinic app to carry out one or more of the functions described elsewhere: data log upload, settings changes, or re-calibration. In the present embodiment, the clinician will upload data from each wearable independently to ensure all past data is captured. The clinic app's user interface will provide simple menus and forms to allow the neurosurgeon user to navigate easily through the functions. The clinic app will further provide data management software to allow the user to review past downloaded data. Graphical trend displays, sorting by date, filtering by event type (example: valve transitions, posture changes), ICP thresholds, total valve ‘open’ time, and alerts according to physician-set thresholds may be included.

If settings changes or re-calibration is performed, the clinician will ensure that the implant and updated wearable carry out a data exchange so that the new settings or calibration data are written to the implant's memory. Alternative embodiments may run on smartphone platforms or the Apple or Linux operating systems. The smartshunt system may utilize a separate, dedicated hardware device to host the clinic app, versus relying on third-party devices.

The patient's wearables may automatically, wirelessly synchronize with one another so that all of them hold the full data log and the latest settings. In this way only one of a patient's wearables needs to be interrogated in the office.

The clinic app may include direct interfaces with third-party electronic health record systems such as Epic to allow upload of smartshunt system data directly to the patient's chart.

Further, the wearable may feature internet connectivity, allowing direct upload of data logs to the patient's electronic medical records (EMR). This could be done directly, or through a cloud database that accepts the data logs and relays relevant data to the EMR application, as well as to a physician portal that can be accessed through any internet browser. This could happen with or without clinician intervention, potentially eliminating the need for the clinic app.

In the embodiment where the wearable automatically uploads data to the cloud, the clinician portal could be configured to send alerts to the physician based on preset criteria for alert thresholds. Alerts could also be issued if patient data is missing, or a battery recharge has been required for a certain period without patient action.

The clinic app may be used by the patient to perform their own data downloads or settings changes. Limits may be placed on settings to prevent unsafe situations, and changes could be automatically communicated to the patient's physician(s) via a web app. The clinic app could employ machine learning or artificial intelligence algorithms to assist the clinician in analyzing trends or to guide therapy based on past and present data.

The smartshunt system may be designed to resemble existing mechanical shunts in form, fit and function (except for the added functionality described above). Its implantation takes place with the same basic steps, with minor modifications:

Pre-operatively and with implant still in sterile packaging, charge the implant's battery using the wireless charger unit. Carry out simple functional checks using the wearable and clinic software/app. The surgeon may then open the scalp tissue to expose the skull surface.

The surgeon may drill two burr holes through the skull. One will accommodate the proximal catheter (diameter 2.5 mm). The other will accommodate the Sensor Cylinder that extends perpendicularly from the valve module (diameter 5 mm).

The surgeon may tunnel through the brain and place the proximal catheter in the ventricle using the same technique previously used with other shunts. A stylet or ventriculoscope may be used to provide stiffness to the proximal catheter during tunneling.

The surgeon may create a pocket in the scalp for the valve module between the periosteum and the galea. For the smartshunt system, the pocket must be positioned such that the 4 mm diameter sensor cylinder can be inserted into its dedicated burr hole.

The surgeon may then access the abdominal peritoneum and pass the distal shunt from the abdomen to the head using the same subcutaneous tunneling techniques used with other shunts.

The surgeon can cut the distal catheter to length and flush the distal catheter with saline and connect to the valve assembly's output fitting, as with commercial shunts. Another step is to cut the proximal catheter to length and flush the proximal catheter with saline and connect to the valve assembly's input fitting, as with other shunts. For the alternative dual-lumen proximal catheter design, both lumens must be flushed separately.

The surgeon or a proxy therefor may perform a final functional check with the wearable and clinic app. The incisions may be closed and recovery for the patient may begin.

Below are several alternative embodiments and variations of the clinic app.

During implantation, there may be an additional step in which a gold-standard commercial ICP sensor is used to verify accuracy of the smartshunt system's pressure reading. If there is a significant offset or gain difference, this could be programmed into the implant as a calibration offset or gain coefficient that must be applied to every future reading.

Prior to implantation, the valve assembly may be pre-heated to body temperature for a period of several minutes or hours in a dedicated container. This is to precondition the pressure sensor to body temperature, ensuring accurate measurements immediately following implantation and prior to any calibration step using a separate reference ICP sensor system. The temperature preconditioning device could be a standard piece of hospital equipment, and may be designed to hold and pre-warm the valve assembly while still in its sterile packaging.

The valve assembly may be packaged together or separate from the catheters in its sterile packaging. The valve assembly could be designed to be compatible with third party catheters by proper design of its barbed fitting attachments.

### Post-Op Training, Calibration and System Check

After patients have recovered from implantation surgery, they may be issued two wearables, a charger, and a dock. Before discharge from the hospital, clinical staff trains the patient to use these devices. The wearables and charger may be paired with the implant, and the functionality of these devices are checked during the training session. Also prior to discharge, the physician may uses the clinic app to calibrate the patient's smartshunt system. This comprises taking at least one ICP reading while the patient is upright, and at least one other ICP reading with the patient recumbent. Pressure delta between upright and recumbent, as well as tilt sensor offsets, are written to the wearable and implant memories, and used in an algorithm to correct for posture in future readings. Recumbent readings may include readings taken in the supine, prone, left/right lateral decubitus, partially recumbent (example 45 degree angle), tilted head (all angles), combinations of head and body positions, or other readings. There may be separate posture calibration parameters measured for each position.

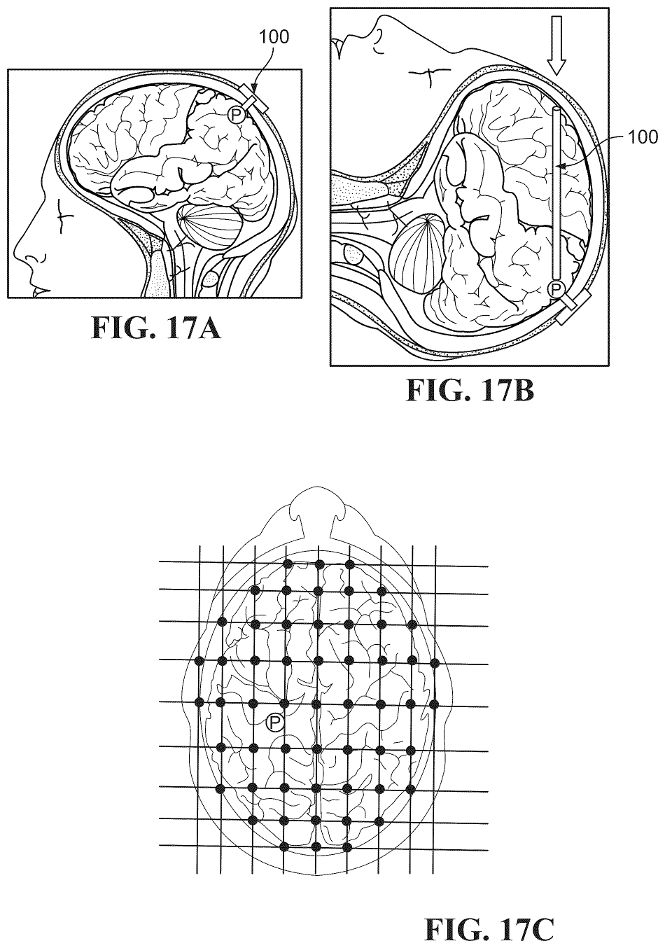

The main purpose of this positional calibration is to determine ICP reading offsets caused by fluid column or brain weight influence on the smartshunt system's pressure sensor for different body postures. This is illustrated in the left and center illustrations in FIGS. 17A-17C.

In another embodiment, the general calibration step described above to offset fluid column/brain weight may have a specific embodiment that involves brain mapping each individual patient's internal cranial volume. The method comprises use of MRI, CT or other imaging technology to create a 3D map of the inner surface of the cranium. The map also defines coordinates for the implanted ICP sensor and 3-axis tilt sensor (shown as “P” in figure). The map could be in the form of a lookup table or matrix of surface coordinates. It could also be a set of 3D vector equations. The map image could be taken just prior to implantation, with the sensor coordinates noted during surgery and added afterwards. Alternatively, the image could be obtained after surgery so that the Implant is included.

After implantation, during each ICP reading, the tilt sensor's three axes are sampled at the same or nearly same time as the ICP sensor is sampled. The implant's processor determines the gravity vector direction based on the tilt sensor samples and calculates the distance along the gravity vector between “P” and the surface of the 3D map of the cranial wall. For each pressure reading, this distance is calculated. The processor may do this by interpolating between surface point coordinates in a lookup table. The processor may calculate the distance based on tilt angles, or the distances may be pre-calculated, and the lookup table populated with a distance for each (x, y, z) tilt vector measured by the tilt sensor.

The implant processor calculates column pressure PFC based on CSF and brain density times column volume (the sensor surface area times the distance above the sensor and below the cranial wall of the segment parallel with the gravity vector). A standard density for {CSF+brain) may be used, or a per-patient density may be used. If the latter, calibration may be done to better determine each patient's {CSF+brain matter} density. This is done noninvasively by wireless readings with the patient's head in several positions where the gravity vector is known (e.g. supine, left/right recumbent, prone, upright). This measurement can be repeated periodically, for example annually, to ensure accuracy as the patient grows.

The processor subtracts column pressure from the measured pressure to obtain accurate ICP.

### Other Post-Op Alternatives and Variations.

In place of a lookup table, mathematical formulas may be used by the processor to calculate the posture offset. The processing may be carried out in part or wholly by an upstream processor, such as the wearable or other processor, rather than in the implant. The implant would upload raw tilt data the higher level processor, and the processor would download results or commands back to the implant.

Rather than generate a 3D map using advanced imagery techniques, a simpler shape for the cranial inner surface can be assumed based on external measurements of the head. A clinician could enter these measurements into a software program, as well as the approximate location of the sensor noted during surgery and relative to anatomical markers. The program would then generate the appropriate lookup table or formulas for posture correction and these would be loaded into the Implant or other processor carrying out the correction calculation. While potentially less precise than a 3D map, this saves on the expensive imaging step and may deliver sufficient accuracy correction.

### Home Use

After discharge from the hospital, the patient must keep at least one charged wearable nearby at all times. The quantitative definition of ‘nearby’ depends on the wireless communications technology used between the implant and wearable—for BTLE systems this is typically approximately 10 meters, less when interfering structures such as walls exist in the communication path. As a safety margin, the instructions for use will require the users to keep the wearable within a specified distance, for example three meters and in line of sight (no walls), at all times. The wearable, which may take the form of a necklace, wristwatch, or clothing attachment, can be removed when sleeping/showering/etc. as long as it is kept within the specified distance. A waterproof version may be developed to allow swimming or related wet activities. A key function of the wearable is to measure ambient pressure and subtract this from the implant's absolute ICP reading to obtain gauge ICP for valve control, data logging, and display to the patient.

At home, the patient must: keep a wearable nearby at all times; keep unused wearables and the charger unit charged by placing them in their dock. The patient may need to ensure the wearable is charged at all times; this generally means docking the device at night, using a portable charger that may be provided, and frequently checking the wearable's battery charge level. The patient will periodically (for example once per day), use the wearable to take an on-demand ICP reading, check for good function, and note any messages or warnings from the implant, including battery life. This is done by hitting a button or other control on the wearable and observing the reading on the wearable's screen or a different device in wireless communication with the wearable, such as a notebook computer, desktop computer, tablet, smartphone or the like.

If the patient receives a ‘low battery’ or ‘recharge due’ message from the implant via the wearable, they must recharge the battery by holding the charger against the head, as close to the valve module as practical. They will typically keep it there for 1-4 hours. A hat or harness may be provided to facilitate charging. The smartshunt system will provide an audible, visual, or haptic indication of charge status. If the system inadvertently becomes completely discharged, it may require some time before it can provide status. If charging is interrupted, it may be resumed later.

On-demand ICP/system status readings may be taken at any time by hitting a control (e.g. pushbutton) on the wearable and observing the screen. Besides ICP and battery state of charge, the system may provide other status indicators for each reading, including “Possible Clogged Shunt”, “Valve State”, “Time of Last Reading”, “Time to Next Reading”, “Other System Alert—contact physician” and “Ambient Pressure Reading”.

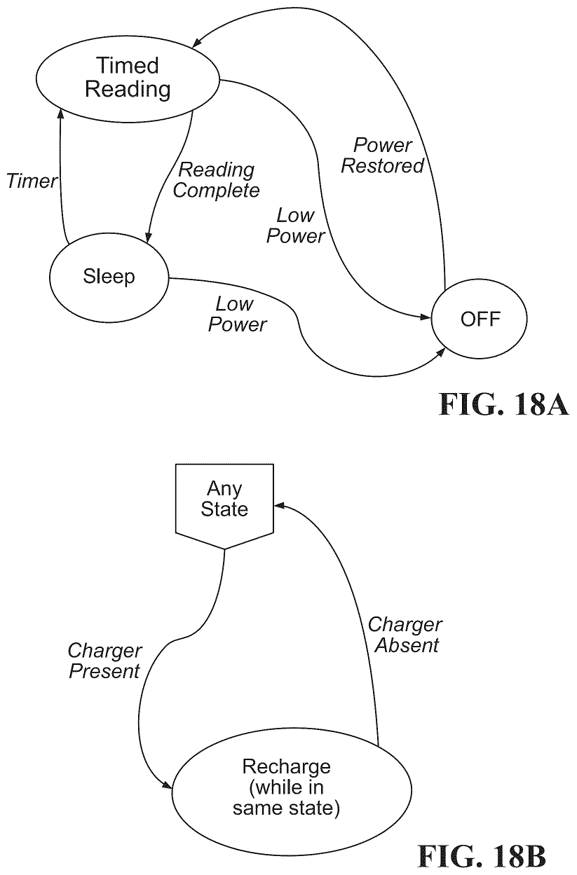

FIGS. 18A and 18B is a state machine for the implant. At the right, the two boxes indicate that the implant's battery recharge function is independent of the state it is in. For any state, when a charger is brought near enough to the implant to generate a sufficiently strong signal, the implant will recharge while continuing to operate within the state machine at left. Should the charger be removed, charging discontinues but the state machine continues to operate as shown. This includes the case where the implant is in the “OFF” state. Details on the states and their transitions are provided below.

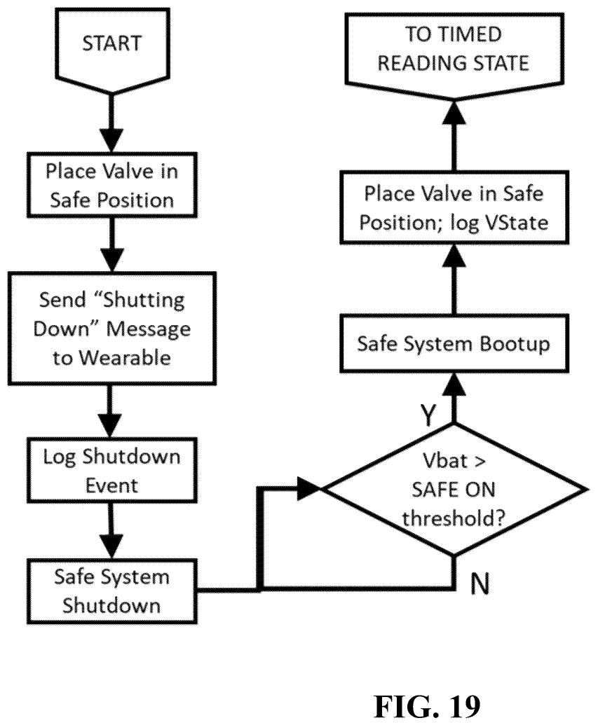

FIG. 19 illustrates the OFF state. This is the state the implant is shipped and stored in, with the battery discharged to a safe shipping/storage level. The implant may also enter OFF state when its battery capacity gets to a low level, but still has enough capacity to transition the valve and send a message to the wearable one more time. As soon as a user brings the charger within range of the implant's antenna, the system should have adequate voltage and the battery should begin charging. The “SAFE ON” threshold for Vbat should be sufficiently high, so that when the system wakes up, it has enough power to boot up, and also enough power to safely shut down once again if the charger is suddenly removed. The “SAFE ON” threshold should be somewhat higher than the “SHUTDOWN” threshold, i.e., hysteresis should be built in, to prevent the system from dithering between off and on. Safe shutdown includes putting the valve into a preset “safe” position—which will be OPEN as a default but may be set to CLOSED by the user during parameters settings. Also prior to shutdown, the implant will attempt, a limited number of times, to alert the wearable that it is shutting down. The wearable would then issue an alert to the user via its display that the implant must be recharged immediately. Safe shutdown also includes gracefully shutting down the system so that nothing is in a random, undefined or unsafe state. Although ‘Vbat’ is used in the figure, battery voltage is just one possible means by which the system can assess remaining battery capacity. Tracking of total output current over time is another method. Others are known to those skilled in the art.

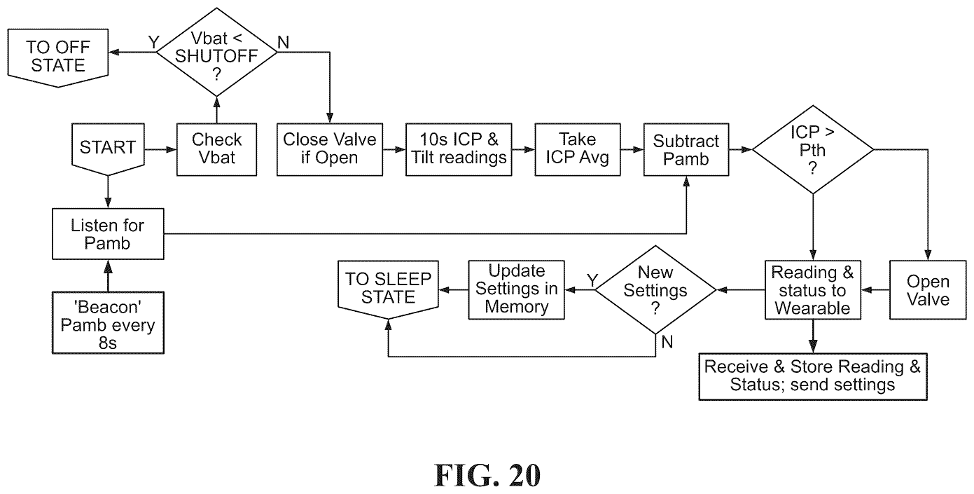

FIG. 20 illustrates implant behavior in the timed reading state. Black boxes are implant actions and blue boxes are wearable actions. The internal implant timer counts down the reading interval period, which is settable using the clinic app. The user may set different intervals for the valve closed state (default 15 minutes) and the valve open state (default 30 seconds), the open state interval being shorter to prevent over drainage. When the timer times out, the system wakes up. The processor first checks battery capacity (shown here as voltage Vbat exceeding a threshold but could also be checked by other means such as current logging or event timing). The processor logs capacity and may calculate estimated time to next recharge—this parameter, along with any alerts related to low battery, will be uploaded to the wearable later in the sequence. If battery capacity is below the ‘SHUTOFF’ threshold, it has just enough capacity to complete the OFF state sequence, and the system will transition to the OFF state. If battery capacity is above SHUTOFF threshold, the system continues by closing the valve (if the last known state was Open) and taking its 10 second reading. The valve is closed during the reading to remove any siphon effect by the distal catheter on the pressure at the sensor.

Tilt readings may be taken alongside the ICP readings to provide correctional offsets due to posture, using the lookup table or other methods described herein. Tilt correction may be done for every ICP sample, or once for a number of ICP samples—that is, the sampling frequency for tilt may be different from the sampling frequency for ICP. In one embodiment, 2e sample absolute ICP at 200 Hz and 3-axis acceleration at 100 Hz, each with its own four-tap rolling average filter so that filtered readings occur at 50 Hz for ICP and 25 Hz for tilt with respect to gravity. Large sample values above reasonable amplitude thresholds are ignored, assumed to be due to short transients in ICP or acceleration. The implant processor uses three-dimensional vector analysis to calculate the patient's head tilt with respect to gravity, applying a correction factor to account for the accelerometer's tilt with respect to the patient's head. This correction factor is measured during post-surgical calibration with the patient sitting upright. Once tilt angle is known, the implant references a lookup table that was loaded into it during the most recent calibration session. The lookup table provides fluid column offset for every possible tilt angle, and is based on pressure measurements made during calibration, as well as fluid column length vectors calculated based on external measurements of the patient's skull made during calibration, as depicted in FIGS. 17A-17C. The processor determines the fluid column offset from the lookup table and applies it to each ICP reading. Since the filtered sampling bandwidth for 50 Hz for ICP and 25 Hz for tilt, each tilt correction factor is applied to two ICP samples in sequence, until the next tilt correction factor is measured.

At the end of the ten-second measurement interval, all the tilt-corrected ICP readings are averaged to obtain a single ICP.

In parallel with the ICP measurement, the implant is listening for the wearable to broadcast the current ambient pressure Pamb, which it may do using the Bluetooth ‘beacon’ mode. The time interval between Pamb broadcasts (8 seconds in the figure) is less than the implant's ICP measurement interval (10 seconds in the figure), to ensure at least one Pamb broadcast is available during the measurement. The implant's processor then subtracts Pamb from the absolute average ICP to obtain gauge average ICP. If average ICP is greater than the user-set pressure threshold Pth for opening, the processor opens the valve and logs its valve status. Otherwise, the valve remains closed. There may be a different Pth for the case where the valve was initially closed versus the case where the valve was initially open. The ‘closed’ threshold Pthc would generally be several mmHg higher than the ‘open’ threshold Ptho. The hysteresis imposed here would prevent the valve from dithering repeatedly between the open and closed states and consuming excessive power.

With the new valve position set, the implant now uploads its ICP reading, plus any other status information and alerts, to the Wearable. Additionally, the Implant may upload any previously logged data that was not previously uploaded; logged events such as shutdown, recharge, watchdog timer or other internal faults; siphon event; or ‘call physician’ for events such as the system or a component such as the valve or battery nearing the end of rated lifetime or rated number of cycles. The implant may also upload its current valve state, its current user settings, and the date-time at which those settings were last updated. The user settings may include Ptho, Pthc, ‘open’ measurement time interval, ‘closed’ measurement time interval, valve ‘safe’ state (open or closed), and ICP averaging window.

The wearable may store the uploaded settings in its data log, which will be downloaded from the wearable by a clinician using the clinic app. If the wearable has settings (downloaded to it previously via the clinic app) with a more recent date-time stamp than the settings in the implant's memory, it downloads these to the implant and these become the new settings, along with their date-time stamp. In this way, the implant always as the latest settings. Once the data and any new settings are exchanged, the implant returns to SLEEP state.

The wearable's beacon function can be read and used by any implant. The other communications between wearable and implant can only take place between a wearable and implant that are designated to communicate with one another, using serial numbers or other pre-programmed codes. This ensures that only one patient's data is kept in each wearable.

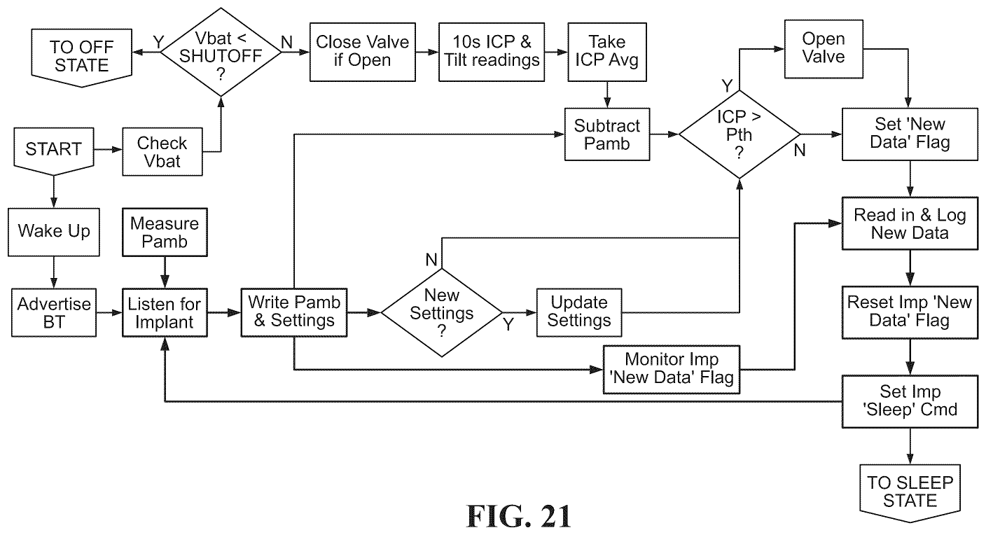

FIG. 21 provides an alternative timed reading algorithm that does not require the wearable to be in beacon mode part of the time and in transmit/receive modes at other times is also shown. Here the wearable is a straightforward Bluetooth central device, and the implant is a peripheral device. The wearable constantly listens for the implant to advertise itself, which it does at the beginning of each timed cycle after waking up. Once the wearable recognizes the implant, it writes its most recent ambient pressure reading to the implant, as well as a timestamped set of settings that were downloaded to the wearable by the clinic app. The implant compares the date/time stamp on the downloaded settings with the date/timestamp of the settings it already has in memory, and writes the new settings if the date/timestamp is newer. The implant acquires and processes its ICP and tilt sensor samples as described elsewhere and subtracts the newly downloaded Pamb to obtain gauge pressure. It compares to the threshold and sets the valve, and sets a ‘New Data Ready’ flag in a designated register to indicate it is ready to upload the new reading. Meanwhile, the wearable has been monitoring the ‘New Data Ready’ register. When it detects new data is ready, it uploads all data stored in designated registers, containing ICP, valve state, and the other system variables described elsewhere. If there is past data from earlier readings that was not uploaded due to loss of contact or some other function, it uploads those as well. There may be error checks or handshakes between the wearable and implant to ensure error-free data upload. When the wearable has received the new data upload, it writes this to its data log and will display the latest data uploaded, along with the time of reading and time to next reading, on its display. The wearable may maintain a real time clock (RTC) to timestamp the newly uploaded data, obviating the need for the implant to have an RTC. Finally, the wearable resets the implant's ‘New Data Ready’ flag back to the negative state, and also sets a register that commands the implant to go to SLEEP state. The wearable then goes back to listening for the implant to advertise during the next measurement cycle.

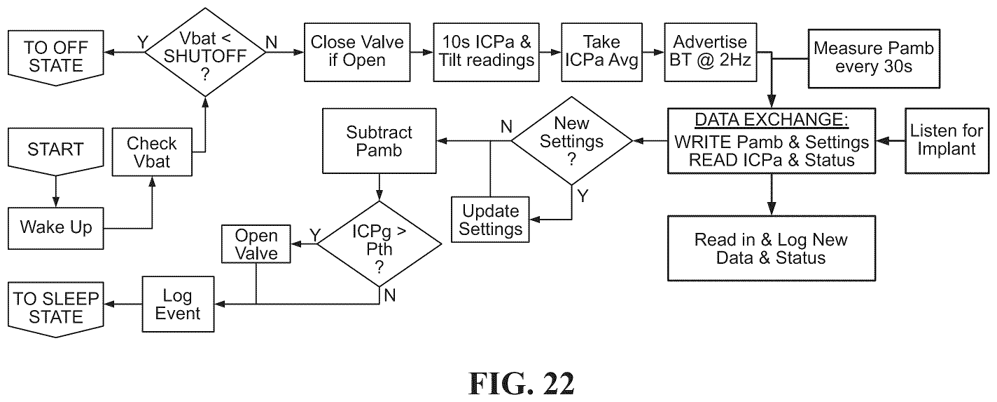

FIG. 22 provides still another alternative flow for the timed reading state. This one minimizes complexity and implant power draw by requiring only one data exchange between the implant and wearable per timed reading. This algorithm relies on the wearable and the implant to each determine derived parameters separately, such as gauge ICP, valve state, valve switching threshold Pth, and time to next reading. During the data exchange, each device must transmit all of its Settings (Pth, window length, time between readings, valve safe state, etc.) to the other along with the date/time stamp for those settings, and each device must determine whether to update its settings to the settings with more recent date/time. An alternative approach would be for the wearable to wait for the implant to determine its derived parameters and write them to memory, and then do a second data exchange where the wearable reads the actual derived parameters from implant registers. This would avoid the failure mode wherein the wearable and implant somehow arrive at different derived parameters. In this alternative, the wearable could wait a certain preset time period, or it could read a “new data ready” flag in the Implant and read in the new derived parameters when ready.

Should an implant's timer time out and the implant does not receive a beacon signal or contact a wearable, it will proceed through the sequence using the last known Pamb, and store its data locally, flagging it as ‘not yet uploaded’. It may also make a log entry stating ‘no wearable contact’. At the next wearable contact, it will upload any readings made without a wearable, including the ‘no wearable contact’ flag.

Each patient may have two or more wearables. Since each one may contain only part of the full data log, the patient may need to bring all wearables to the clinic for full datalog upload using the clinic app. Other embodiments of the smartshunt system may include a synchronization function between wearables when they are in proximity to one another, for example on the dock.

If a patient's wearables are synchronized, they can issue a visual or audible alert when no wearable has contacted the patient's implant over a designated time period, for example one hour. This function may also be carried out using the dock as an intermediary device.

Aspects of the three alternative timed reading algorithms presented here can be deleted, added, or combined to create more alternative versions of the algorithm, as would be obvious to one skilled in the art.

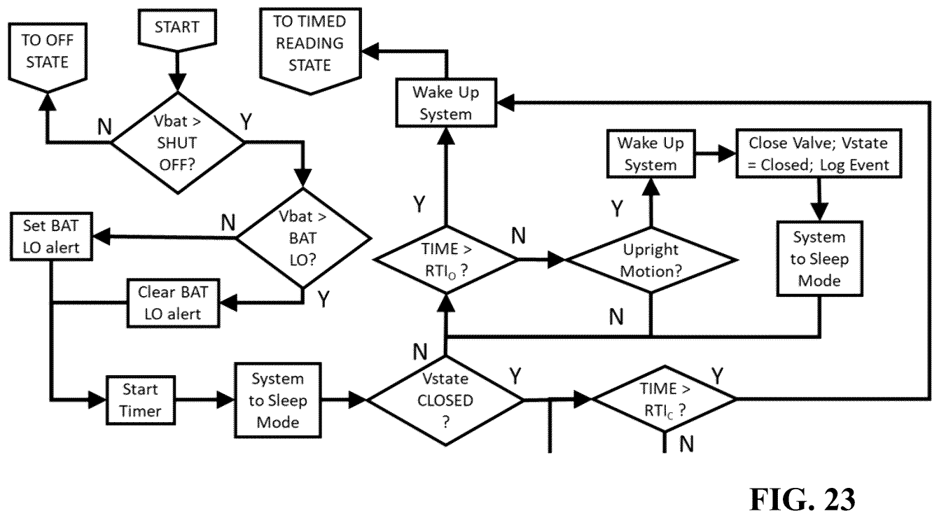

FIG. 23 illustrates the implant behavior in the SLEEP state. Prior to entering the low-power sleep mode, the system does a final battery capacity check. If the battery is very low (below the SHUTOFF threshold) the system enters the OFF state. If it is low enough to trigger a user alert, it sets a “low battery” flag to provide an alert to the wearable during the next timed reading. It then starts its timer and places all devices into their low power ‘Sleep’ mode. Depending on valve state, the timer will either count down to the RTIO interval for valve open, or the RTIC interval for valve closed. These intervals are user settable, and default to 30 seconds (open) and 15 minutes (closed). In the valve open case, the tilt sensor's low-power motion detect function is enabled, which keeps the tilt sensor in a low power state but issues a signal to the processor if motion above a certain threshold is detected. This is to sense the case in which the patient moves from a recumbent to an upright position with respect to gravity, potentially causing a siphoning event. Should this occur, the smartshunt system immediately wakes up, closes the valve to prevent drainage during the siphon event, logs the event in memory, and returns the system to sleep mode. When the timer finally counts down to its appropriate RTI interval, the system wakes up and enters the TIMED READING state.

### Home Use Alternative Embodiments

In an embodiment, as described elsewhere in this specification, the smartshunt system may use an internal body pressure (e.g. peritoneal) as a surrogate for ambient pressure reading. In this case, it is not required to have a wearable nearby at all times. In this alternative, there would be an ‘External Reader’ to read and display the ICP. The reader would not be worn at all times and would not require its own ambient pressure sensor, although it could have one to improve the accuracy of the ICP reading. It could also use its ambient P sensor to re-calibrate the downstream pressure sensor in the implant that is reading IAP as a surrogate for Pamb. As with all embodiments, the external unit and charger could be separate devices or combined into a single device.

In yet another alternative embodiment and as an added feature, the smartshunt system may provide an additional external device that could manually force the valve to change to the open or closed state, regardless of the state of charge of the implant battery. It would generally be for emergency use when the implant battery is depleted and the valve must change state immediately to prevent potential harm to the patient. The device could inductively couple a strong magnetic field at a given frequency into the implant, similar to the action of the charger. This wireless power signal would be at a frequency different from the implant's charging and communications frequencies. An internal filter in the implant would direct the energy at that frequency exclusively to a drive circuit that would apply the necessary current (ac or dc) required to change the valve to the desired state. Different frequencies could be used for an ‘open’ or ‘close’ signal. None of the coupled current would go to other circuits in the implant. The system could share one of the other antennas (communications or charging), or have its own antenna. The external device providing the signal could be a separate device, or could be combined with the wearable, the charger, or the non-wearable external unit. For example, the charger could be configured to provide three frequencies: normal charging, override open, and override closed.

Alternatively, other ways than unique frequency could differentiate the signal in order to bypass other circuits and exclusively operate the valve. A digital signal, based on passive RFID technology, could be encoded to operate only the valve. Internal circuitry would wake up and use the signal power to operate a simple logic register, timer, and receiver that would match a bit code from the external device to a hard-coded code in memory, in order to activate a switch that sends power to the valve. The incoming power signal could be modulated with the bit code. Other ways, such as an acoustic or vibrational signal that actuates a MEMS device to switch the power through to the valve could be employed.

In yet another embodiment, the wearable logs data from each timed reading and reports the most recent ICP and other parameters when queried by the patient via a pushbutton, or when data is downloaded by the clinic device. Likewise new settings are only changed when the timed reading is triggered by the implant. Instead of the implant triggering the timed reading and data exchange, the wearable could trigger the reading immediately. This would require the implant to ‘listen’ for the wearable and possible consume more power than in the baseline methods. However, it would allow the user to get the latest ICP without having to wait for the implant timer to complete its cycle.

In yet another embodiment, during calibration, when the physician is wirelessly reading the patient's ICP while placing them in different positions, it may be inconvenient to wait for the next ICP reading to be triggered by the implant timer (default in valve-closed state is 15 minutes per reading). A wearable-triggered reading as described above would solve this, possibly at the expense of additional power draw in the implant due to the need to advertise to the wearable or listen for advertising frequently. Instead, the physician could simply use the wearable to set the timer for closed or open valve readings to a very short interval, for example 10 seconds. This could be done with the system described in the baseline architecture.

### Clinic Visits

Most hydrocephalus patients are recommended to visit their physician's office periodically. At regular intervals, for example annually, the patient's visit will include wireless checks and recalibration of the system. Calibration methods, settings changes, and the like are described above.

### Explanation and Maintenance

If there are questions about the smartshunt system's function or accuracy, invasive means such as an external ICP device may be employed to check the smartshunt system's ICP accuracy and proper functional switching. A non-invasive alternative may involve injecting the implant's reservoir with a needle and measuring ICP pressure using a sensor upstream of the needle, or a small ICP sensor as are known in the art inserted through a large bore needle.

The smartshunt system is designed to allow invasive inspection and maintenance with only minimal impact. For example, the valve assembly can be simply detached and replaced if the battery or valve assembly is near its the end of its maximum rated cycle lifetime, or if degraded function is suspected for any other reason. The surgeon must open the scalp, disconnect the proximal and distal catheters at the barbed fittings. The valve assembly can be removed and replaced as a single unit. While disconnected, the proximal or distal catheter may be inspected in situ using a ventriculoscope or may be flushed or cleaned using a stylet or other means.

Software or firmware upgrades for the valve assembly may be carried out noninvasively using the wireless interface. This may be done using the clinic app. The upgrade could also be carried out remotely using the internet.

### Miscellaneous Additional Embodiments & Features

A variety of additional embodiments and features are possible, as alternatives to or in combination with the concepts presented above. These are presented below.

Direct ΔP Switching. The main embodiment calculates gauge ICP to control the valve by subtracting an ambient pressure measurement provided by the wearable. An alternative embodiment uses Intra Abdominal Pressure (IAP) as a surrogate for ambient pressure, and uses one-time calibration to subtract out IAP added by the body. A further alternative to these is to include two pressure sensors, one upstream and the other downstream of the valve, as in the ‘IAP as surrogate’ approach. The valve would simply be switched on ΔP across the two sensors, effectively assuming that IAP measured equals ambient pressure, and thus operating as present delta-pressure valves do. This could be done in combination with an anti-siphon correction based on a tilt sensor, with an existing gravity driven anti-siphon device, or without it.

The Implant may maintain its own real-time clock (RTC) using local or universal time. The implanted RTC could be updated during communications with the wearable, charger, ExU, or other external device, including cloud-based devices in some embodiments. In other architectures, the implant does not require an RTC, but conversion between the implant's clock and real time could be carried out on the appropriate external device.

Detection of Present Valve Position. The processor will use the valve state (Vstate) parameter to keep track of the present state of the latching valve-open or closed. In general, this is based on the processor remembering the last command given to the valve, either via separate connections for the open and closed state or using different polarity for the applied voltage or current. It would be advantageous, however, to have a way to independently verify the present valve state, as a check on the ‘last command issued’ data. Depending on how the valve is constructed, a way for valve state verification could comprise one of: measurement of resistance across the valve's electrical input connectors; a reflective or light blocking indicator on the valve that changes position or reflectivity when the valve changes state, and then draws no power after the valve latches, and a circuit that uses a light source and an optical detector to determine the location of the reflector or light blocker; for valves containing permanent magnets, a hall effect or other magnetic sensor that senses magnetic field changes when the valve's state changes; a proximity sensor outside the valve that, for example a capacitive sensor, that wirelessly detects position of a movable conductive component inside the valve, and other means known in the art.

Algorithm for Clog Detect Early Warning. An algorithm in the clinic software analyzes all historical data from a patient's data log. Each time the valve is open, it calculates and records the time it takes to reduce ICP across a certain delta (mmHg/min). It does this for every valve open event, calculates a time constant, and stores this. Longitudinally, if time to reduce ICP is increasing over weeks/months/years, it could indicate the shunt's flow resistance is increasing gradually over time. This could trigger a warning or alert to the physician that the smartshunt system is gradually heading for a clog. If flow resistance seems to be decreasing over time, it could mean that something in the smartshunt system is changing and alerts the physician that an investigation may be warranted. As an alternative, the implant, external unit or wearable processor, or a processor in the cloud, could track this data and perform the algorithm.

End of Rated Life Alerts. The implant could track number of valve transitions, battery charge cycles, and other limited life item parameters in the system and issue alerts to the user when a valve assembly change will be required due to device age or cycle time. instead of the implant, the wearable, clinic app, external unit, or a cloud database could execute this algorithm.

Algorithm to minimize valve transitions and extend battery life. In the one-sensor baseline embodiment, one may wish to sample ICP frequently when the valve is open to prevent overdrainage—as a default every 30 seconds. However, such frequent sampling will deplete the battery faster, since the valve must be closed during ICP sampling to prevent the distal shunt's fluid column from affecting the ICP reading. For worst-case patients who drain frequently the battery discharge rate may be unacceptable. So instead of simply sampling every 30 seconds until ICP falls below the ‘valve closed’ threshold, the smartshunt system may sample only twice after opening, once at 30 seconds and again at 60 seconds. The smartshunt system then determines what the reduction in ICP was in the first 30 seconds. This ICP reduction rate is then extrapolated to estimate the length of drainage time required to reduce ICP to below the safe threshold. The smartshunt system would then leave the valve open and refrain from sampling ICP until after the estimated drainage time has elapsed. Alternatively, the smartshunt system could sample ICP at a point before the estimated time has elapsed (for example at one-half or two-thirds of the estimated time), calculate a new drainage rate and time estimate to completion, and refrain from sampling until the new time interval is elapsed or nearly elapsed. Obviously, the specific time intervals mentioned here can be changed.

Algorithm to check ‘sanity’ of pressure readings. The smartshunt system may sample ICP many times and average over a predefined measurement interval, default value 10 seconds, to obtain a single average ICP reading. The readings may be corrected by a tilt offset as described herein. The smartshunt system may improve the accuracy of the average by rejecting samples that are clearly too high or too low in pressure, with thresholds determined by analysis of the physiological range of ICP, even during transients. For example, the smartshunt system may reject all samples below −10 and above 30 mmHg gauge, or it may reject transient events whose amplitude exceeds the window average by more than two standard deviations, and that are shorter than a certain time, for example 5 milliseconds. The smartshunt system could reject samples for which the tilt measurement is too high or low compared to a “physically reasonable” threshold for normal activity. The smartshunt system may also determine the standard deviation of a portion or all of the samples taken during the interval, and reject the entire reading if standard deviation exceeds a preset threshold. The smartshunt system could also reject the entire reading if too much variation in tilt is observed throughout the reading interval, as determined comparison of standard deviation to a threshold. The ‘entire reading rejection’ could occur after analysis at the end of the interval, or in mid-interval if a certain amount of ‘bad data time’ occurs. If the entire reading is rejected, the smartshunt system may immediately start over and take an entirely new reading. The smartshunt system may log the failed reading for future analysis of the data log.

Ambient Pressure Out of Range. The smartshunt system can detect an ICP value that is out of range of its measurement capability, either from the ambient pressure reading from the wearable or the ICP reading from the implant (in alternate embodiments from the downstream distal implant sensor or the external unit). In this case, it may set the valve to its designated ‘safe state’, log the event, and continue with timed readings until ICP and ambient pressure are back in measurement range. If the wearable detects an ambient pressure that is out of system range, it may send a visual or audible alert to a user to relocate to a place with higher pressure (generally, lower altitude). If the implant detects an ICP that is too high or low, it may alert the user via the wearable.

Valve open during Timed Reading. It may not be necessary to close the valve for timed readings taken during a drainage event, when the valve is open. If the ICP sensor is located in the parenchyma, far from the drainage point in the ventricle, the system may not register a pressure change significant enough to affect valve operation.

In addition to each implanted patient going home with at least 2 wearables-one to wear while the other recharges, the smartshunt system may comprise a phone app that can perform the wearable function for the patient and their family members. This is for the case where a patient misplaces or breaks their wearable and has no backup. The phone app is publicly available so anyone can download it for use with the smartshunt system. The user can decide when to activate and de-activate the phone app. In one embodiment, the phone app makes the phone behave just like the wearable.

In another embodiment, the phone app is very simple and does not require pairing with any specific implant. It simply measures and broadcasts the ambient pressure every several seconds in a Bluetooth “beacon” mode. “Beacon” mode is a newer Bluetooth mode where the devices do not have to pair or connect in the classic way. One device just broadcasts an ID number and some data and any device within range can receive the data and take action if the ID number is a certain value. In this embodiment, the implant software does its usual timed wakeup, then scans for its designated wearable. If it finds its wearable, it continues as usual. If after some time it does not find its wearable, it listens for the ‘beacon’ signal. The beacon signal contains a special ID number that every implant will know. If it finds a beacon, it uses the beacon's ambient pressure for the current reading cycle. If it does not find a beacon, it uses its last known ambient pressure and carries out the reading. So the “lines of defense” for getting the ambient pressure correction are:
Get ambient pressure from the dedicated wearable (normal case); if not, then
Get ambient pressure from a beacon from any nearby smart device that has our app on it and has a pressure sensor; if not, then
Get ambient pressure from the last valid reading obtained
If (ii) or (iii) is used, the implant data log should note this for that reading.

Further algorithms may include a ‘sanity check’ on ICP change for case (iii) above. If there has been a very large change in the implant's ICP reading over a short period of time, the implant may use assume ambient pressure has changed a lot but the patient's ICP has not. In this case the implant uses Pamb(n)=ICPa(n)−ICPg(n−1), where Pamb(n) is the ambient pressure used for the current valve state decision, ICPa(n) is the current absolute ICP reading, and ICPg(n−1) is the gauge ICP from the last reading where a valid Pamb was obtained. ‘Valid’ could mean the last reading from an outside source (i or ii above). It could also mean the last reading from iii above where the reading did not violate the pre-defined ‘sanity’ limits. So as an example:
Normal timed ICP reading, but implant cannot contact a wearable (i) or a beacon (ii).
Reading uses the last valid Pamb (iii above) to obtain the present ICPg=gauge ICP, which will determine valve state.
But the newly calculated ICPg fails a sanity test, because it shows an increase of >60 mmHg since the last reading 15 minutes ago. Such an increase in that period is not deemed physiologically likely. It is assumed it happened because Pamb changed by 50 mmHg or more since that time.
d. So the smartshunt system uses ICPa(n)-ICPg(n−1) as its ambient P. It switches the valve based on its new ICPg and notes the sanity event in the data log.

What has been described above includes examples of the present specification. It is, of course, not possible to describe every conceivable combination of components or methodologies for purposes of describing the present specification, but one of ordinary skill in the art may recognize that many further combinations and permutations of the present specification are possible. Each of the components described above may be combined or added together in any permutation to define embodiments disclosed herein. Accordingly, the present specification is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A cranial shunt implantable into a patient, the cranial shunt comprising:
a first catheter;
a second catheter;
a valve assembly operatively coupled with the first and second catheters, the valve assembly comprising an inlet and a microcontroller, wherein the first catheter transfers cerebrospinal fluid (CSF) to the inlet;
a pressure sensor configured to provide intracranial pressure (ICP) of the patient to the microcontroller; and
a tilt sensor configured to provide an angle of orientation relative to gravity of a cranium of the patient to the microcontroller to provide correctional offsets due to posture of the patient to the microcontroller;
wherein the valve assembly passes or blocks the CSF flow to the second catheter based on instructions from the microcontroller.

2. The cranial shunt of claim 1 further comprising an external charging device and a power subsystem, wherein the power subsystem comprises a rechargeable electromechanical energy storage cell and a power management circuit for wireless charging by the external charging device.

3. The cranial shunt of claim 1 further comprising a control subsystem and a power subsystem, wherein the microcontroller sends status data of the power subsystem to the control subsystem.

4. The cranial shunt of claim 3 further comprising a communication subsystem, wherein the control subsystem sends commands and data to and receives data from, the communications subsystem.

5. The cranial shunt of claim 4 further comprising an external device, wherein the communications subsystem communicates wirelessly with the external device.

6. The cranial shunt of claim 1 further comprising a first antenna and a second antenna.

7. The cranial shunt of claim 6 further comprising an external charging device and a power subsystem, wherein the power subsystem comprises a rechargeable electromechanical energy storage cell.

8. The cranial shunt of claim 7 wherein the first antenna resides on a printed circuit board (PCB) or within a component inside an electronics box and the second antenna is a single loop track that encircles a perimeter of the valve assembly.

9. The cranial shunt of claim 8, wherein the first and second antennas operate at spaced frequencies so charging of the rechargeable electromechanical energy storage cell and communication functions of the microcontroller can take place simultaneously for uninterrupted operation during charging of the rechargeable electromechanical energy storage cell.

10. The shunt of claim 9, wherein a charging frequency of the rechargeable electromechanical energy storage cell and any subharmonics resulting from charging are outside a range of human hearing.

11. The cranial shunt of claim 1, wherein the pressure sensor is attached to a tube, and measures intra-cranial pressure in a subarachnoid space within the cranium, or inside a brain's parenchyma.

12. The cranial shunt of claim 1, wherein the pressure sensor measures ICP at a location outside a fluid channel connected to the valve assembly.

13. The cranial shunt of claim 12, wherein if the valve assembly is open for a specified time and ICP fails to decrease by a preselected threshold amount, an external device alerts the patient the first catheter is clogged.

14. The cranial shunt of claim 1 further comprising an external device, wherein the external device provides an ambient pressure reading to the microcontroller.

15. The cranial shunt of claim 14, wherein the microcontroller uses the ambient pressure reading to adjust the ICP based on the patient's environmental surroundings.

16. The cranial shunt of claim 14, wherein the external device comprises a wearable device.

17. The cranial shunt of claim 16, wherein the wearable device comprises a wrist worn device.

18. A medical device comprising:
an implanted catheter;
an implanted valve assembly operatively coupled with the catheter, the valve assembly comprising an inlet and a microcontroller, wherein the catheter transfers cerebrospinal fluid (CSF) to the inlet;
an implanted pressure sensor configured to provide intracranial pressure (ICP) of a patient to the microcontroller, wherein the valve assembly passes or blocks the CSF based on instructions from the microcontroller;
an implanted rechargeable electromechanical energy storage cell operatively coupled with the microcontroller;
an external charging device configured to charge the implanted rechargeable electromechanical energy storage cell wirelessly through derma;
an external communication device in wireless data communication with the microcontroller, wherein the external communication device is configured to receive and send information from and to the microcontroller;
an external docking station configured to charge rechargeable batteries located within said external charging device and said external communication device; and
a clinical software application installed on a device, the clinical software application in communication with the external communication device.

19. The medical device of claim 18, wherein the information received and sent by the external communication device comprises ambient pressure of an environment of the patient.

20. The medical device of claim 18, wherein the external communication device provides alerts indicating a potentially undesirable condition for the patient, wherein such alerts are communicated to the external communication device wirelessly from the microcontroller.

21. The medical device of claim 20, wherein the potentially undesirable condition comprises a blockage in the catheter.

22. The medical device of claim 18, wherein the external communication device provides a measurement of ambient pressure to the microcontroller.

23. The medical device of claim 18, wherein the external communication device is configured to be stored on the external docking station.

24. The medical device of claim 18, wherein the microcontroller uses am ambient pressure reading to adjust the ICP based on environmental surroundings of the patient.

25. The medical device of claim 18, wherein the external communication device comprises a wearable device.

26. The medical device of claim 25, wherein the wearable device comprises a wrist worn device.

27. The medical device of claim 18 further comprising a tilt sensor configured to provide an angle of orientation relative to gravity of a cranium of the patient to the microcontroller to provide correctional offsets due to posture of the patient to the microcontroller.
